(19) United States
(12) Patent Application Publication
(10) Pub. No.: US 2002/0155582 A1
(43) Pub. Date: Oct. 24, 2002

(54) Replication competent hepatitis C virus and methods of use

(76) Inventors: Stanley M. Lemon, Galveston, TX (US); MinKyung Yi, Galveston, TX (US)

Correspondence Address: MUETING, RAASCH & GEBHARDT, P.A., P.O. BOX 581415, MINNEAPOLIS, MN, 55458, US

(21) Appl. No.: 09/747,419

(22) Filed: Dec. 23, 2000

Related U.S. Application Data
(60) Provisional application No. 60/171,909, filed on Dec. 23, 1999.

(51) Int. Cl. A61K 48/00; C12N 7/01; C12N 15/861; C07H 21/04; A01N 63/00; A61K 39/42; C12N 7/00; C12N 15/86

(52) U.S. Cl. 435/235.1; 424/93.21; 435/456; 536/23.72; 424/147.1; 424/159.1; 536/24.3; 536/24.33

(57) ABSTRACT

The present invention provides a replication competent hepatitis C virus that includes a heterologous polynucleotide present in the 3′ non-translated RNA. The invention also includes methods for modifying a hepatitis C virus, selecting a replication competent hepatitis C virus, detecting a replication competent hepatitis C virus, and identifying a compound that inhibits replication of a hepatitis C virus.

### CONTINUING APPLICATION DATA

This application claims the benefit of U.S. Provisional Application Serial No. 60/171,909, filed Dec. 23, 1999, which is incorporated by reference herein.

### GOVERNMENT FUNDING

The present invention was made with government support under Grant No. U19-AI40035 awarded by the NIH. The Government has certain rights in this invention.

### BACKGROUND

Hepatitis C virus (HCV) is the most common cause of chronic viral hepatitis within the United States, infecting approximately 4 million Americans and responsible for the deaths of 8,000-10,000 persons annually due to progressive hepatic fibrosis leading to cirrhosis and/or the development of hepatocellular carcinoma. HCV is a single stranded, positive-sense RNA virus with a genome length of approximately 9.6 kb. It is currently classified within a separate genus of the flavivirus family, the genus Hepacivirus. The HCV genome contains a single large open reading frame (ORF) that follows a 5′ non-translated RNA of approximately 342 bases containing an internal ribosome entry segment (IRES) directing cap-independent initiation of viral translation. The large ORF encodes a polyprotein which undergoes post-translational cleavage, under control of cellular and viral proteinases. This yields a series of structural proteins which include a core or nucleocapsid protein, two envelope glycoproteins, E1 and E2, and at least six nonstructural replicative proteins. These include NS2 (which with the adjacent NS3 sequence demonstrates cis-active metalloproteinase activity at the NS2/NS3 cleavage site), NS3 (a serine proteinase/NTPase/RNA helicase), NS4A (serine proteinase accessory factor), NS4B, NS5A, and NS5B (RNA-dependent RNA polymerase).

With the exception of the 5′ non-translated RNA, there is substantial genetic heterogeneity among different stains of HCV. Phylogenetic analyses have led to the classification of HCV strains into a series of genetically distinct “genotypes,” each of which contains a group of genetically related viruses. The genetic distance between some of these genotypes is large enough to suggest that there may be biologically significant serotypic differences as well. There is little understanding of the extent to which infection with a virus of any one genotype might confer protection against viruses of a different genotype.

Several types of human interferon have proven effective in the treatment of infection by HCV, either alone as monotherapy, or in combination with ribavirin. However, treatment with interferon-ribavirin carries a high risk of treatment failure, either primary failure of virus elimination, or relapse of the infection upon cessation of therapy. Moreover, these therapeutic agents are relatively toxic and are associated with a high frequency of adverse reactions. The development of better (more effective and safer) antiviral agents capable of suppressing or eliminating HCV infection has been hindered by the fact that this virus replicates with very low efficiency, or not at all, in cultured cells. The absence of a highly permissive cell culture system that is capable of supporting robust replication of the virus has prevented the development of high throughput antiviral screens for use in the development of inhibitors of viral replication, and has delayed the investigation of the virus and relevant aspects of its molecular and cellular biology. It has also stymied efforts at vaccine development and the immunologic characterization of the virus, the human response to HCV, and the diseases associated with infection. The development of infectious molecular cDNA clones of the viral genome has done little to solve this problem, since virus can be rescued from the RNA transcribed from such clones only by its injection into the liver of a living chimpanzee or other susceptible primate.

### SUMMARY OF THE INVENTION

The present invention represents a significant advance in the study of hepatitis C virus (HCV). The 3′ non-translated RNA of HCV includes three distinct regions; the variable region, the poly U-UC region, and the conserved region. The 3′ non-translated RNA is believed to have secondary structure that is critical for RNA replication of the RNA. It was previously shown that mutants of an HCV genotype 1a infectious cDNA clone containing deletions of some regions in the 3′ non-translated RNA had lost the ability to infect chimpanzees. Deletion of the variable region of the 3′ non-translated RNA did not alter the in vivo infectivity of HCV in chimpanzees, thus showing that the secondary structure of the 3′ non-translated variable region was not critical. However, the insertion of additional nucleotides into the variable region would be expected to potentially alter the secondary structure throughout the 3′ non-translated RNA, and thereby potentially reduce in vivo infectivity. It was found that a coding region could be inserted into an infectious clone of HCV such that the infectivity of the viral RNA and ability to replicate and generate infectious virus particles was retained, and the coding region was expressed.

Accordingly, the present invention is directed to a replication competent HCV that includes an HCV genome and a heterologous polynucleotide. Optionally, the HCV can be isolated. The HCV genome inlcudes a 3′ non-translated RNA and the heterologous polynucleotide is present in the 3′ non-translated RNA, for instance the variable region of the 3′ non-translated RNA. The heterologous polynucleotide can include a coding sequence, which can encode, for instance, a selectable marker, a detectable marker, an immunogenic polypeptide, or a fusion polypeptide. The selectable marker can encode resistance to an antibiotic including, for instance, neomycin or phleomycin D1. A regulatory region, for instance an internal ribosome entry site, can be operably linked to the coding sequence. The coding sequence can encode a transactivator, for instance a tat polypeptide. The tat polypeptide can include SEQ ID NO:19.

Optionally, the replication competent HCV can include a first coding sequence encoding a transactivator, a second coding sequence encoding a selectable marker, and a third coding sequence encoding a cis-active proteinase. The third coding sequence is located between the first and second coding sequences, and the first, second, and third coding sequences can together encode a fusion polypeptide. The cis-active proteinase can include amino acids SEQ ID NO:30.

The replication competent HCV may replicate in vitro in a human cell, for instance a cultured cell, including a hepatocyte. The replication competent HCV may replicate in vivo. The replication competent HCV can have a genotype of, for instance, 1a, 1b, 1c, 2a, 2b, 2c, 3a, 3b, 4, 5a, of 6a.

The replication competent HCV can be an RNA polynucleotide, and optionally present in a viral particle. Alternatively, the replication competent HCV can be a DNA polynucleotide, and optionally present in a vector.

The invention is also directed to a cell that includes a replication competent HCV, including a cultured cell.

Further provided by the invention is a method for modifying an HCV that includes a 3′ non-translated RNA. The method includes inserting a heterologous polynucleotide into the 3′ non-translated RNA.

The invention is also directed to a method for selecting a replication competent HCV. The method includes incubating a first cell in the presence of a selecting agent. The cell includes an HCV that includes a heterologous polynucleotide, for instance present in the variable region of the 3′ non-translated RNA, that includes a coding sequence encoding a selectable marker that confers resistance to the selecting agent. The selecting agent inhibits replication of a cell that does not express the selectable marker. The method further includes detecting the presence or absence of a cell that replicates in the presence of the selecting agent, wherein the presence of such a cell indicates the HCV is replication competent.

The method can further include isolating a virus particle produced by the first cell, exposing a second cell to the isolated virus particle and incubating the second cell in the presence of the selecting agent, and detecting the presence or absence of a second cell that replicates in the presence of the selecting agent, wherein the presence of such a cell indicates the HCV present in the first cell produces an infectious virus particle. The invention includes the isolated virus particle. Isolating the virus particle can include removing cells from media in which the first cell is incubated.

The invention is also directed to a method for detecting a replication competent HCV. The method includes incubating a cell that includes an HCV, where the HCV includes a heterologous polynucleotide that includes a coding sequence encoding a transactivator. The heterologous polynucleotide can be present in the variable region of the 3′ non-translated RNA. The cell includes a transactivated coding region and an operator sequence operably linked to the transactivated coding region, and the transactivated coding region encodes a detectable marker, for instance secretory alkaline phosphatase. The presence or absence of the detectable marker is detected, and the presence of the detectable marker indicates the cell comprises a replication competent HCV. Optionally, the replication competent HCV can be isolated from the cell.

The heterologous polynucleotide can further include a coding sequence encoding a selectable marker, and the coding sequence encoding the transactivator and the coding sequence encoding the selectable marker together encode a fusion polypeptide. Optionally, the heterologous polynucleotide can further include a coding sequence that encodes a cis-active proteinase present between the coding sequence encoding the selectable marker and the coding sequence encoding the transactivator. Optionally, the HCV can further include a regulatory region, for instance an internal ribosome entry site, operably linked to the coding sequences encoding the fusion polypeptide. The transactivator can include a tat polypeptide, including, for instance, SEQ ID NO:19. The fusion polypeptide can further include a cis-active proteinase.

The invention further provides a method for identifying a compound that inhibits replication of an HCV. The method includes contacting a cell that includes a replication competent HCV with a compound. The replication competent HCV includes a heterologous polynucleotide. The cell is inclubated under conditions allowing replication of the replication competent HCV in the absence of the compound. The presence or absence of the replication competent HCV is identified. A decrease in the presence of replication competent HCV in the cell contacted with the compound relative to the presence of replication competent HCV in a cell not contacted by the compound indicates the compound inhibits replication of the replication competent HCV.

The heterologous polynucleotide can include a first coding sequence that encodes a transactivator, and the cell can include a polynucleotide that includes a transactivated coding sequence encoding a detectable marker and an operator sequence operably linked to the transactivated coding sequence. The transactivator can bind to the operator sequence and alter expression of the transactivated coding sequence. The presence of replication competent HCV in the cell is identified by detecting the detectable marker encoded by the transactivated coding sequence. The replication competent HCV can be introduced to the cell before contacting the cell with the compound, and the cell can be contacted with the compound before, at the same time, or after introducing the replication competent HCV to the cell.

The invention is also directed to a vector that includes a cDNA of a replication competent HCV. The HCV includes an HCV genome and a heterologous polynucleotide. The HCV genome includes a 3′ non-translated RNA, and the heterologous polynucleotide is present in the 3′ non-translated RNA. The heterologous polypeptide can include a coding sequence, and can further include a regulatory region, for instance an internal ribosome entry site, operably linked to the coding sequence. The vector can also include a promoter operably linked to the replication competent HCV. Also provided by the invention is an RNA molecule produced by the vector, where the RNA molecule includes the replication competent HCV, and a cell that includes the vector.

Definitions

As used herein, the term “replication competent” refers to an HCV that replicates in vitro or in vivo. As used herein, the term “replicates in vitro” indicates the HCV replicates in a cell that is growing in culture. The cultured cell can be one that has been selected to grow in culture, including, for instance, an immortalized or a transformed cell. Alternatively, the cultured cell can be one that has been explanted from an animal. “Replicates in vivo” indicates the HCV replicates in a cell within the body of an animal, for instance a primate (including a chimpanzee) or a human. Unless noted otherwise, replication in a cell includes the synthesis of viral nucleic acid, for instance synthesis of the negative-sense strand, and the production of infectious viral particles, i.e., viral particles that can infect a cell and result in the production of more infectious viral particles.

Unless otherwise noted, as used herein, the term “replication competent HCV” refers to an RNA sequence of the positive-sense genome RNA, the complement thereof (i.e., the negative-sense RNA), and the DNA sequences corresponding to the positive-sense and the negative-sense RNA sequences. The positive-sense genome RNA of a replication competent HCV can be present in a virus particle.

As used herein, “HCV genome” refers to a nucleotide sequence that is typically present in a wild type HCV, for instance a clinical isolate. An HCV genome includes nucleotide sequences corresponding to a 5′ non-translated RNA and a 3′ non-translated RNA, and nucleotide sequences encoding a viral polyprotein, which is subsequently processed to yield polypeptides C, E1, E2, P7, NS2, NS3, NS4A, NS4B, NS5A, and NS5B. Preferably, a replication competent HCV includes an intact polyprotein encoding each of these polypeptides, and the polyprotein does not include nucleotides that are from other viruses.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences and/or non-translated regions. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. The term “heterologous polynucleotide” refers to a polynucleotide that has been inserted into the HCV genome, typically by using recombinant DNA techniques, and is not naturally occurring.

The term “3′ non-translated RNA” and “3′ non-translated region” are used interchangeably, and are terms of art. The term refers to the nucleotides that are at the 3′ end of the positive-sense strand of the HCV, the complement thereof (i.e., the negative-sense RNA), and the corresponding DNA sequences of the positive-sense and the negative-sense RNA sequences. The 3′ non-translated RNA includes, from 5′ to 3′, nucleotides of variable length and sequence (referred to as the variable region), a poly-pyrimidine tract (the poly U-UC region), and a highly conserved sequence of about 100 nucleotides (the conserved region) (see FIG. 2). The variable region begins at the first nucleotide following the stop codon of the NS5B coding region, and ends immediately before the nucleotides of the poly U-UC region. The poly U-UC region is a stretch of predominantly U residues, CU residues, or C(U)n-repeats. When the nucleotide sequence of a variable region is compared between members of the same genotype, there is typically a great deal of similarity; however, there is typically very little similarity in the nucleotide sequence of the variable regions between members of different genotypes (see, for instance, Yamada et al., Virology, 223, 255-261 (1996)). The length of the variable region can vary.

A “coding region” or “coding sequence” is a nucleotide region that encodes a polypeptide and, when placed under the control of appropriate regulatory sequences, expresses the encoded polypeptide. The boundaries of a coding region are generally determined by a translation start codon at its 5′ end and a translation stop codon at its 3′ end. A coding region can encode one or more polypeptides. For instance, a coding region can encode a polypeptide that is subsequently processed into several polypeptides. A regulatory sequence or regulatory region is a nucleotide sequence that regulates expression of a coding region to which it is operably linked. Nonlimiting examples of regulatory sequences include promoters, transcription initiation sites, translation start sites, internal ribosome entry sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

As used herein the term “marker” refers to a molecule, preferably a polypeptide. The polynucleotide that encodes the marker is referred to as a “marker sequence.” A “selectable marker” is a polypeptide that inhibits a compound, for instance an antibiotic, from preventing cell growth. A “detectable marker” is a polypeptide that can be detected. A marker can be both selectable and detectable.

“Polypeptide” as used herein refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like.

As used herein, an “immunogenic polypeptide” refers to a polypeptide which elicits an immunological response in an animal. An immunological response to a polypeptide is the development in a subject of a cellular and/or antibody-mediated immune response to the polypeptide. Usually, an immunological response includes but is not limited to one or more of the following effects: the production of antibodies, B cells, helper T cells, suppressor T cells, and/or cytotoxic T cells, directed specifically to an epitope or epitopes of the polypeptide fragment.

As used herein a “fusion polypeptide” refers to a polypeptide encoded by a coding region that is made up of two coding regions that have been joined together in frame such that the two coding regions now encode a single polypeptide.

As used herein, a “transactivator” is a polypeptide that affects in trans the expression of a transactivated coding region. A “transactivated coding region” is a coding region to which is operably linked a regulatory sequence referred to herein as an “operator sequence.” As used herein, the term “operator sequence” is a type of regulatory region and includes a nucleotide sequence to which a transactivator can bind to alter expression of an operably linked transactivated coding region.

An “isolated” virus means a virus that has been removed from its natural environment. For instance, a virus that has been removed from an animal is an isolated virus. Another example of an isolated virus is one that has been removed from the cultured cells in which the virus was propagated, for instance by removing media containing the virus. Preferably, a virus of this invention is purified, i.e., essentially free from any other associated cellular products or other impurities. The term “purified” is defined as encompassing preparations of a virus having less than about 50%, more preferable less than about 25% contaminating associated cellular products or other impurities.

As used herein, the phrase “selecting a replication competent hepatitis C virus” refers to identifying a cell that includes a replication competent HCV under conditions that prevent the replication of cells that do not include a replication competent HCV.

As used herein, the term “introduce” and “introducing” refer to providing an HCV to a cell under conditions that the HCV is taken up by the cell in such a way that the HCV can then replicate. The HCV can be a virus particle, or a nucleic acid molecule, preferably RNA.

Unless otherwise specified, “a,” “an,” “the,” and “at least one” are used interchangeably and mean one or more than one.

### BRIEF DESCRIPTION OF THE FIGURES

FIG. 1. Genomic organization of MK0-Z, ds-MK0-Z, and 3′ETZ. The rightward facing arrows, location and direction of transcription initiation; 5′NTR, 5′non-translated RNA; C, core protein; E1, envelope protein 1; E2, envelope protein 2; E2-p7, a polypeptide of about 7 kDa; NS2, non-structural protein 2; NS3, non-structural protein 3; NS4A, non-structural protein 4A; NS4B, non-structural protein 4B; NS5A, non-structural protein 5A; NS5B, non-structural protein 5B; EMCV IRES, encephalomyocarditis virus internal ribosome entry site; tat, portion of the human immunodeficiency virus I (HIV I) tat protein; 2A, 2A proteinase of foot-and-mouth disease virus (FMDV); Zeo, polypeptide encoding resistance to phleomycin; 3′NTR, 3′ non-translated RNA.

FIG. 2. Site of insertion of heterologous sequence within the 3′NTR (3′ non-translated RNA) of H77C strain (pCV-H77C). Variable region, poly U-UC, and Conserved region refer to regions of the 3′ non-translated RNA; EMCV IRES, tat, FMDV 2A, and Zeo, see legend to FIG. 1; NS5B refers to the last 12 nucleotides that encode NS5B.

FIG. 3. Schematic depicting release of SEAP from a reporter cell line by expression of Tat from a modified HCV RNA. EMCV, tat, 2A, and Zeo, see legend to FIG. 1; HIV-LTR, HIV I long terminal repeat transcriptional regulator; SEAP, secretory alkaline phosphatase.

FIG. 4. SEAP activity in medium collected from cells following transfection with RNAs. (A) Huh7-SEAP-o10 cells. (B) Huh7-SEAP-N7 cells. The smaller graph A and B each depict days 1 and 6, but use different scales. Mock, cells exposed to transfection conditions but not RNA; 3′ETZ, MK0-Z, and dS-MK0-Z, the constructs shown in FIG. 1; y-axis, units of secretory alkaline phosphatase activity measured by luminescent signal detected by a TD-20/20 Luminometer (Turner Design, Sunnyvale, Calif.).

FIG. 5. The passage history of two Huh-SEAP-o10 cell sublines (MK0-Z.C-A and MK0-Z.C-B) that were infected with MKO-K and the secretory alkaline phosphatase (SEAP) activity in supernatant media collected at approximately weekly intervals from both surviving cell lines. dSma (C-A) and dSma (C-B) are two Huh-SEAP-o10 cell sublines infected with supernatant fluids collected from cells transfected in parallel with dS-MK0-Z (NS5B-deletion mutant) RNA. Split, points at which the cultures were split are indicated by arrows. The top panel shows the timing and magnitude of Zeocin selection pressure (top panel, mg/ml).

FIG. 6. SEAP expression profiles of Huh-SEAP-o10 cells. (A) Absolute SEAP activities of supernatant media from cells inoculated with supernatant fluids of C-A and C-B MK0-Z infected cell lines. “l1” inoculum=media from C-A subline, “I4” inoculurn=media from C-B subline. None=mock infections. (B) SEAP activity relative to SEAP activity of mock-infected control Huh-SEAP-o10 cells (lost during Zeocin selection).

FIG. 7. LightCycler RT-PCR detection of viral RNA in supernatant fluids of C-A and C-B cells. The plot demonstrates the melting curves of the fluorescence resonance energy transfer signal from products generated from the cell culture samples and associated controls. Fluorescence-d[F2/F1]/dT, the melting curve as calculated by the LightCycler thermal cycler.

FIG. 8. TaqMan RT-PCR detection of HCV RNA in C-A and C-B cell culture supernatants.

FIG. 9. Nucleotide sequence of MK0-Z (SEQ ID NO:17). The initiation codon of the viral polyprotein which undergoes post-translational cleavage is the ATG at nucleotides 342-344. The initiation codon of the inserted heterologous polynucleotide is the ATG at nucleotides 9907-9909.

FIG. 10. Nucleotides 342-10,803 of SEQ ID NO:17, and the polyprotein (SEQ ID NO:20). The amino acid sequence (SEQ ID NO:21) encoded by the heterologous polynucleotide (i.e., nucleotides 9907-10,602 of SEQ ID NO:17) is also shown.

FIG. 11. The results of Taqman RT-PCR of a chimpanzee inoculated with MK0-Z RNA. The term ge/ml refers to genomic equivalents per milliliter.

FIG. 12. Nucleotide sequence of HIVSEAP (SEQ ID NO: 18).

### DETAILED DESCRIPTION OF THE INVENTION

Hepatitis C Virus

The present invention provides hepatitis C viruses (HCV) that include a heterologous polynucleotide inserted into the HCV genome. Preferably, the HCV are replication competent. Preferably the HCV are isolated, more preferably, purified. Unless otherwise noted, HCV genome and other terms that refer to all or a part of an HCV genome (including, for instance, “3′ non-translated RNA”) include an RNA sequence of the positive-sense genome RNA, the complement thereof (i.e., the negative-sense RNA), and the DNA sequences corresponding to the positive-sense and the negative-sense RNA sequences.

It is expected that HCV from different sources, including molecularly cloned laboratory strains, for instance cDNA clones of HCV genomes, and clinical isolates can be used in the methods described below to yield replication competent HCV of the present invention. Examples of molecularly cloned laboratory strains include the HCV that is encoded by pCV-H77C (Yanagi et al., Proc. Natl. Acad. Sci., USA, 94, 8738-8743 (1997)), and pHCV-N as modified by Beard et al. (Hepatol., 30, 316-324 (1999)). Clinical isolates can be from a source of infectious HCV, including tissue samples, for instance from blood, plasma, serum, liver biopsy, or leukocytes, from an infected animal, including a human or a primate.

It is expected that the HCV of the present invention are not limited to a specific genotype. For instance, an HCV of the present invention can be genotype 1a, 1b, 1c, 2a, 2b, 2c, 3a, 3b, 4, 5a, or 6a (as defined by Simmons, Hepatology, 21, 570-583 (1995)). It is also expected that HCV used in the methods described below can be prepared by recombinant, enzymatic, or chemical techniques. Typically, an HCV that is modified as described herein to include a heterologous polynucleotide is able to replicate in vivo, preferably in a chimpanzee, prior to inserting the heterologous polypeptide. Methods for determining whether an HCV is able to replicate in a chimpanzee are described herein.

Preferably, the nucleotide sequence of an HCV used in the methods of the present invention is similar to the nucleotide sequence of an HCV, preferable an HCV of genotype 1a, 1b, 2a, or 2b. An example of an HCV of genotype 1a is present at Genbank accession AF011751. Examples of an HCV of genotype 1b are present at Genbank accession AF139594 or at Genbank accession AJ238799. An example of an HCV of genotype 2a is present at Genbank accession AF238481. An example of an HCV of genotype 2b is present at Genbank accession AB030907. The similarity is referred to as structural similarity and is determined by aligning the residues of the two polynucleotides (i.e., the nucleotide sequence of the candidate HCV and the nucleotide sequence of the HCV of genotype 1a, 1b, 2a, or 2b) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate HCV is the nucleotide sequence being compared to the nucleotide sequence of the HCV of genotype 1a, 1b, 2a, or 2b. Preferably, two nucleotide sequences are compared using the Blastn program, version 2.0.11, of the BLAST 2 search algorithm, as described by Tatusova, et al. (FEMS Microbiol Lett 1999, 174:247-250), and available at ncbi.nlm.nih.gov/gorf/b12.html. Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on. In the comparison of two nucleotide sequences using the BLAST search algorithm, structural similarity is referred to as “identities.” Preferably, a polynucleotide includes a nucleotide sequence having a structural similarity with the coding region of the HCV of genotype 1a, 1b, 2a, or 2b of at least about 66%, more preferably at least about 77%, most preferably at least about 91% identity.

The heterologous polynucleotide is inserted in the HCV genome, preferably in the HCV 3′ non-translated RNA, more preferably in the variable region of the 3′ non-translated RNA. In one aspect of the invention, the heterologous polynucleotide is inserted into the variable region such that the variable region is not removed. Alternatively, deletions of the variable region can be made, in whole or in part, and replaced with the heterologous polynucleotide. Preferably, in some aspects of the invention, when the HCV has the genotype 1a, more preferably, the strain H77C, the heterologous polynucleotide is inserted in the variable region between nucleotides 5 and 6 of the sequence 5′ CUCUUAAGC 3′, where the sequence shown corresponds to the positive-strand.

The heterologous polynucleotide can include a non-coding region and/or a coding region, preferably a coding region. The coding region can encode a polypeptide including, for instance, a marker, including a detectable marker and/or a selectable marker. Examples of detectable markers include secretory alkaline phosphatase, green fluorescent protein, and molecules that can be detected by antibody. Examples of selectable markers include molecules that confer resistance to antibiotics, including the antibiotics kanamycin, ampicillin, chloramphenicol, tetracycline, neomycin, and formulations of phleomycin D1 including, for example, the formulation available under the trade-name ZEOCIN (Invitrogen, Carlsbad, Calif.). Other examples of polypeptides that can be encoded by the coding region include a transactivator, and/or a fusion polypeptide. Preferably, when the polypeptide is a fusion polypeptide, the coding region includes nucleotides encoding a marker, more preferably, nucleotides encoding a fusion between a transactivator and a marker. Optionally, the coding region can encode an immunogenic polypeptide.

A transactivator is a polypeptide that affects in trans the expression of a coding region, preferably a coding region integrated in the genomic DNA of a cell. Such coding regions are referred to herein as “transactivated coding regions.” The cells containing transactivated coding regions are described in detail herein in the section “Methods of use.” Transactivators useful in the present invention include those that can diffuse within a cell from the cytoplasm into the nucleus to interact with a regulatory region, preferably an operator sequence, that is operably linked to a transactivated coding region. As used herein, the term “operator sequence” is a type of regulatory region and includes a nucleotide sequence to which a transactivator can bind to alter expression of an operably linked transactivated coding region. As used herein, the term “transactivator” includes polypeptides that bind to an operator sequence and either prevent transcription from initiating at, or activate transcription initiation from, a coding region operably linked to the operator sequence. Preferably, a transactivator binds to an operator sequence. Examples of useful transactivators include the HIV tat polypeptide (see, for example, the polypeptide SEQ ID NO:19, which is encoded by nucleotides 5377 to 5591 and 7925 to 7970 of Genbank accession number AF033819). Other useful transactivators include human T cell leukemia virus tax polypeptide (which binds to the operator sequence tax response element, Fujisawa et al., J. Virol, 65, 4525-4528 (1991)), and transactivating polypeptides encoded by spumaviruses in the region between env and the LTR, such as the bel-1 polypeptide in the case of human foamy virus (which binds to the U3 domain of these viruses, Rethwilm et al., Proc. Natl. Acad. Sci. USA, 88, 941-945 (1991)). Alternatively, a post-transcriptional transactivator, such as HIV rev, can be used. HIV rev binds to a 234 nucleotide RNA sequence in the env gene (the rev-response element, or RRE) of HIV (Hadzopolou-Cladaras et al., J. Virol., 63, 1265-1274 (1989)).

Active analogs or active fragments of a transactivator can be used in the invention. An active analog or active fragment of a transactivator is one that is able to bind to an operator sequence and either prevent transcription from initiating at, or activate transcription initiation from, a coding region operably linked to the operator sequence.

Active analogs of a transactivator include polypeptides having amino acid substitutions that do not eliminate the ability to bind to an operator and alter transcription. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, aspartate, and glutamate. The positively charged (basic) amino acids include arginine, lysine, and histidine. The negatively charged (acidic) amino acids include aspartic acid and glutamic acid. Examples of preferred conservative substitutions include Lys for Arg and vice versa to maintain a positive charge; Glu for Asp and vice versa to maintain a negative charge; Ser for Thr so that a free —OH is maintained; and Gln for Asn to maintain a free NH₂.

Active fragments of a transactivator include a portion of the transactivator containing deletions or additions of one or more contiguous or noncontiguous amino acids such that the resulting transactivator will alter expression of an operably linked transactivated coding region. A preferred example of an active fragment of the HIV tat polypeptide includes amino acids amino acids 1-48 of SEQ ID NO:19.

In those aspects of the invention where the heterologous polynucleotide includes a coding region that encodes a fusion polypeptide, the fusion polypeptide can further include amino acids corresponding to a cis-active proteinase. When the fusion polypeptide is a fusion between a transactivator and a marker, preferably the fusion polypeptide also includes amino acids corresponding to a cis-active proteinase. Preferably the amino acids corresponding to a cis-active proteinase are present between the amino acids corresponding to the transactivator and the marker. A cis-active proteinase in this position allows the amino acids corresponding to the transactivator and the marker to be physically separate from each other in the cell within which the HCV is present. Examples of cis-active proteinases that are useful in the present invention include the cis-active 2A proteinase of foot-and-mouth disease (FMDV) virus (see, for example, U.S. Pat. No. 5,846,767 (Halpin et al.) and U.S. Pat. No. 5,912,167 (Palmenberg et al.)), ubiquitin (see, for example, Tauz et al., Virology, 197, 74-85 (1993)), and the NS3 recognition site GADTEDVVCCSMSY (SEQ ID NO:31) (see, for example, Lai et al., J. Virol., 74, 6339-6347 (2000)).

Active analogs and active fragments of cis-active proteinases can also be used. Active analogs of a cis-acting proteinase include polypeptides having amino acid substitutions that do not eliminate the ability of the proteinase to catalyze cleavage. Active fragments of a cis-active proteinase include a portion of the cis-active proteinase containing deletions or additions of one or more contiguous or noncontiguous amino acids such that the resulting cis-active proteinase will catalyze the cleavage of the proteinase.

Optionally and preferably, when the heterologous polynucleotide is present in the 3′ non-translated RNA and includes a coding region, it further includes a regulatory region that is operably linked to the coding region. Preferably, a regulatory region located 5′ of the operably linked coding region provides for the translation of the coding region. A replication competent HCV that has a coding region in the 3′ non-translated RNA is dicistronic.

A preferred regulatory region located 5′ of an operably linked coding region is an internal ribosome entry site (IRES). An IRES allows a ribosome access to mRNA without a requirement for cap recognition and subsequent scanning to the initiator AUG (Pelletier, et al., Nature, 334, 320-325 (1988)). An IRES is located upstream of the translation initiation codon, e.g., ATG or AUG, of the coding sequence to which the IRES is operably linked. The distance between the IRES and the initiation codon is dependent on the type or IRES used, and is known to the art. For instance, poliovirus IRES initiates a ribosome translocation/scanning process to a downstream AUG codon. For other IRES elements, the initiator codon is generally located at the 3′ end of the IRES sequence. Examples of an IRES that can be used in the invention include a viral IRES, preferably a picornaviral IRES or a flaviviral IRES. Examples of poliovirus IRES elements include, for instance, poliovirus IRES, encephalomyocarditis virus IRES, or hepatitis A virus IRES. Examples of preferred flaviviral IRES elements include hepatitis C virus IRES, GB virus B IRES, or a pestivirus IRES, including but not limited to bovine viral diarrhea virus IRES or classical swine fever virus IRES. Other IRES elements with similar secondary and tertiary structure and translation initiation activity can either be generated by mutation of these viral sequences, by cloning of analogous sequences from other viruses (including picornaviruses), or prepared by enzymatic synthesis techniques.

The size of the heterologous polynucleotide is not critical to the invention. It is expected there is no lower limit on the size of the heterologous polynucleotide. It is expected that there is an upper limit on the size of the heterologous polynucleotide. This upper limit can be easily determined by a person skilled in the art, as heterologous polynucleotides that are greater than this upper limit adversely affect replication of an HCV. In increasing order of preference, the heterologous polynucleotide is at least about 10 nucleotides, at least about 20 nucleotides, at least about 30 nucleotides, most preferably at least about 40 nucleotides.

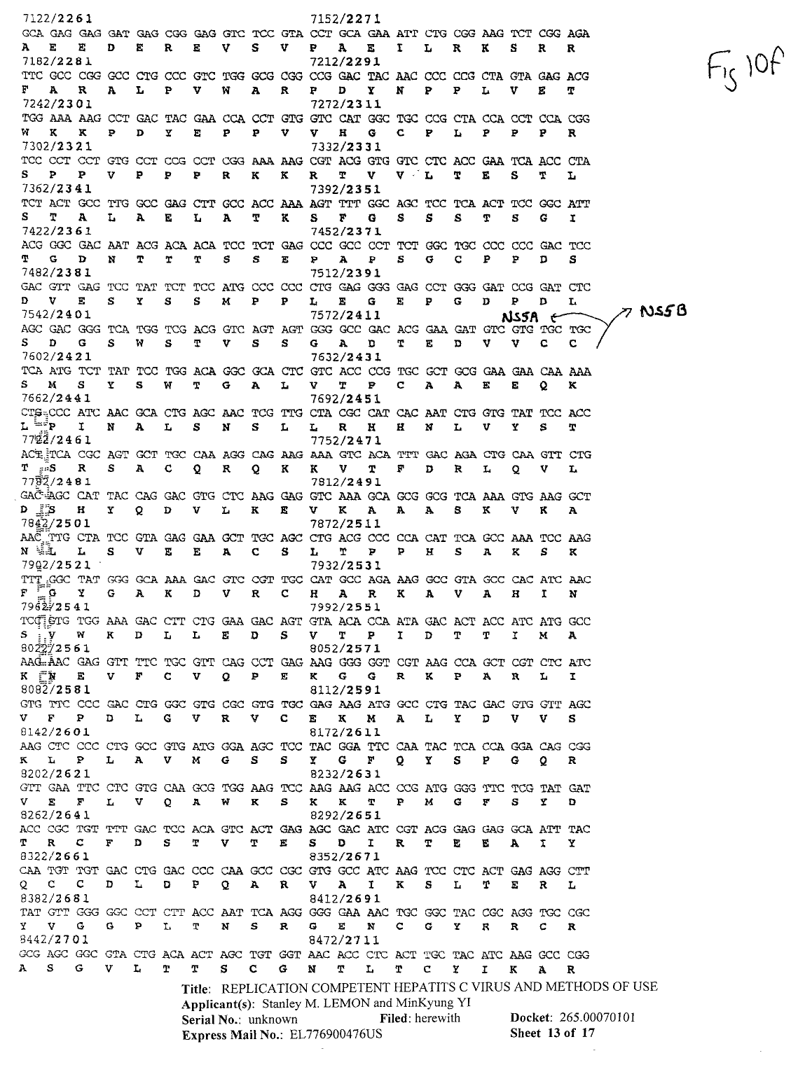

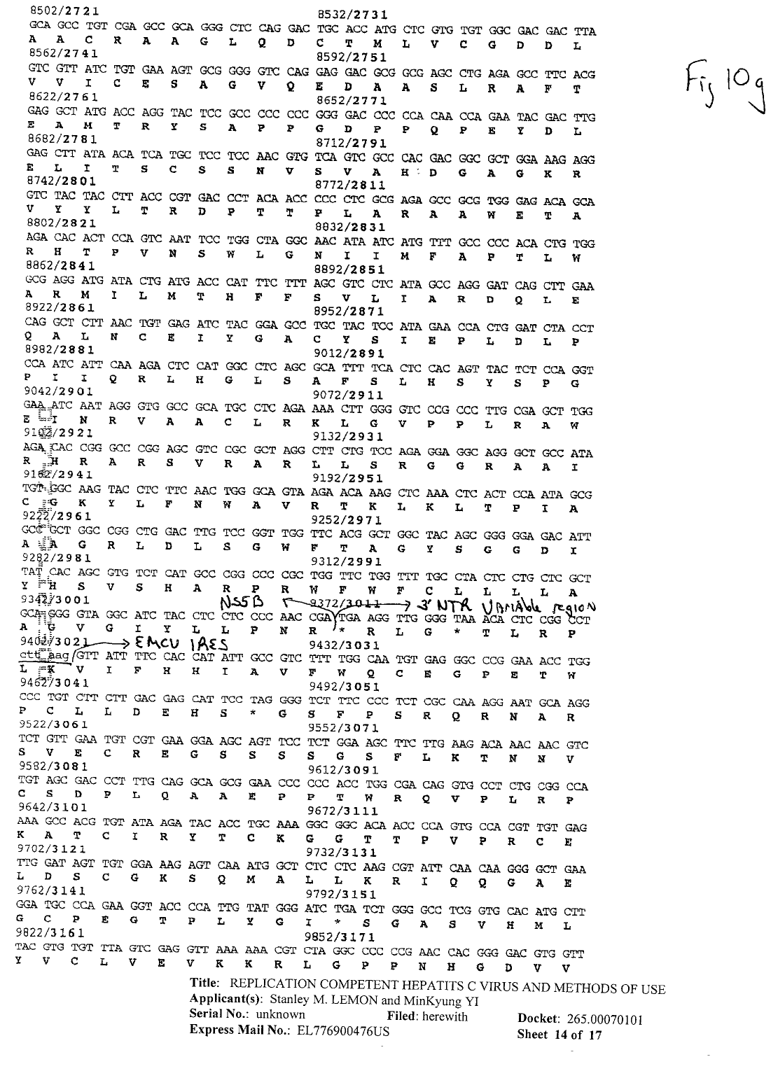

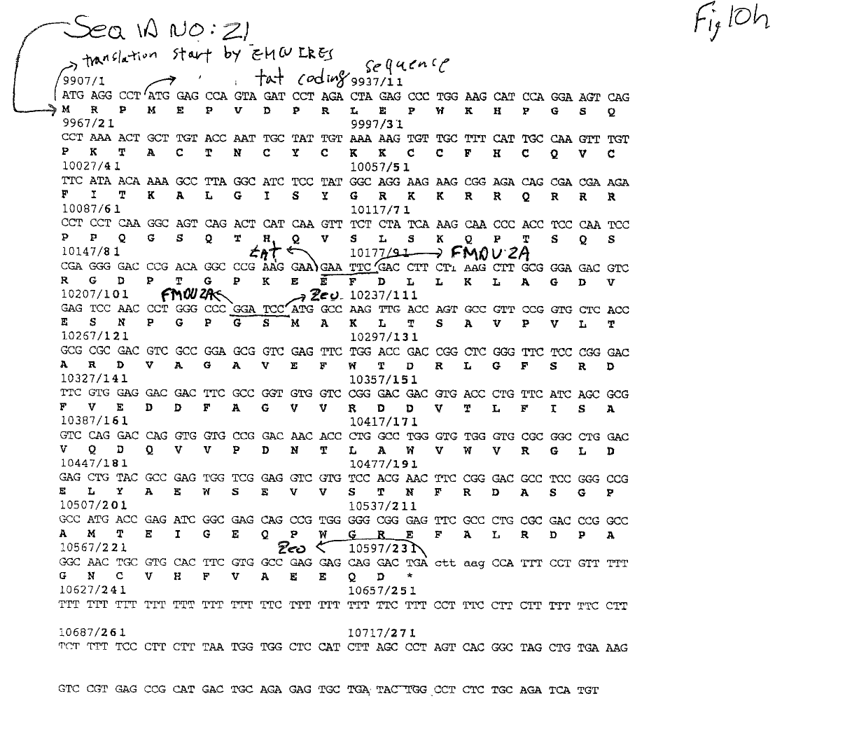

A preferred example of an HCV of the present invention is shown in FIGS. 9 and 10. It should be noted that while SEQ ID NO:17 is a DNA sequence, the present invention contemplates the corresponding RNA sequence, and RNA and DNA complements thereof, as well.

The replication competent HCV of the invention can be present in a vector. When a replication competent HCV is present in a vector the HCV is DNA, including the 5′ non-translated RNA and the 3′ non-translated RNA. Methods for cloning an HCV and inserting it into a vector are known to the art (see, e.g., Yanagi et al., Proc. Natl. Acad. Sci., USA, 94, 8738-8743 (1997); and Rice et al., (U.S. Pat. No. 6,127,116)). Such constructs are often referred to as molecularly cloned laboratory strains, and an HCV that is inserted into a vector is typically referred to as a cDNA clone of the HCV. If the RNA encoded by the HCV is able to replicate in vivo, the HCV present in the vector is referred to as an infectious cDNA clone. A vector is a replicating polynucleotide, such as a plasmid, phage, cosmid, or artificial chromosome to which another polynucleotide may be attached so as to bring about the replication of the attached polynucleotide. A vector can provide for further cloning (amplification of the polynucleotide), i.e., a cloning vector, or for expression of the polypeptide encoded by the coding region, i.e., an expression vector. The term vector includes, but is not limited to, plasmid vectors, viral vectors, cosmid vectors, or artificial chromosome vectors. Preferably the vector is a plasmid. Preferably the vector is able to replicate in a prokaryotic host cell, for instance Escherichia coli. Preferably, the vector can integrate in the genomic DNA of a eukaryotic cell.

An expression vector optionally includes regulatory sequences operably linked to the HCV such that the HCV is transcribed to produce RNA molecules. These RNA molecules can be used, for instance, for introducing an HCV to a cell that is in an animal or growing in culture. The invention is not limited by the use of any particular promoter, and a wide variety are known. Promoters act as regulatory signals that bind RNA polymerase in a cell to initiate transcription of a downstream (3′ direction) HCV. The promoter used in the invention can be a constitutive or an inducible promoter. A preferred promoter for the production of HCV is T7 promoter.

Methods of Use

The present invention is directed to methods for identifying a replication competent HCV, including detecting and/or selecting for cells containing a replication competent HCV. Typically, the cells used in this aspect of the invention are cells growing in culture. Useful cultured cells will support the replication of the HCV of the present invention, and include primary human or chimpanzee hepatocytes, peripheral mononuclear cells, cultured human lymphoid cell lines (for instance lines expressing B-cell and T-cell markers such as Bjab and Molt-4 cells), and continuous cell lines derived from such cells, including Huh-7, HepG2, PH5CH-8. The cells may be primate or human cells, preferably human cells. In general, useful cells include those that support replication of HCV RNA by HCV, including, for instance, replication of the HCV encoded by pCV-H77C. A preferred cultured cell is HuH-7, which is known to workers in the field of HCV (see, for instance, Lohmann et al., Science, 285, 570-574 (1999).

In some aspects of the invention, the cultured cell includes a polynucleotide that includes a coding region, the expression of which is controlled by a transactivator. Such a coding region is referred to herein as a “transactivated coding region.” A transactivated coding region encodes a marker, preferably a detectable marker. Typically, a cultured cell that includes a polynucleotide having a transactivated coding region is used in conjunction with an HCV that includes a coding region encoding a transactivator.

The polynucleotide that includes the transactivated coding region can be present integrated into the genomic DNA of the cell, or present as part of a vector that does not integrate. Preferably, the polynucleotide is integrated into the genomic DNA of the cell. Methods of modifying a cell to contain an integrated DNA are known to the art. An example of making such a cell is described in Example 3.

Operably linked to the transactivated coding region is an operator sequence, i.e., a nucleotide sequence to which a transactivator can bind to alter expression of an operably linked transactivated coding region. The binding of a transactivator can decrease transcription or increase transcription of the operably linked transactivated coding region. In those aspects of the invention where a transactivator increases transcription, preferably there is low transcription of the transactivated coding region in the absence of a transactivator, more preferably, essentially no transcription. An operator sequence can be present upstream (5′) or downstream (3′) of a transactivated coding region. An operator sequence can be a promoter, or can be a nucleotide sequence that is present in addition to a promoter.

In some aspects of the present invention, the replication of cultured cells is inhibited by a selecting agent. Examples of selecting agents are antibiotics, including kanamycin, ampicillin, chloramphenicol, tetracycline, neomycin, and formulations of phleomycin D1. A selecting agent can act to prevent replication of a cell while the agent is present and the cell does not express a molecule that provides resistance to the selecting agent. Alternatively and preferably, a selecting agent can act to kill a cell that does not express a molecule that provides resistance to the selecting agent. Typically, the molecule providing resistance to a selecting agent is expressed in the cell by an HCV that includes a heterologous polynucleotide. Alternatively, the molecule providing resistance to a selecting agent is expressed by the cell but the expression of the molecule is controlled by an HCV that is present in the cell. The concentration of the selecting agent is chosen such that a cell that does not contain a molecule providing resistance to a selecting agent does not replicate. The appropriate concentration of a selecting agent varies depending on the particular selecting agent, and can be easily determined by one having ordinary skill in the art using known techniques.

When a polynucleotide that includes a replication competent HCV is introduced into a cell that is growing in culture, the polynucleotide can be introduced using techniques known to the art. Such techniques include, for instance, liposome and non-liposome mediated transfection. The Examples describe the use of one type of liposome mediated transfection. Non-liposome mediated transfection methods include, for instance, electroporation.

Preferably, when a replication competent HCV is identified using cultured cells, its ability to replicate is verified by introducing the HCV to a cell present in an animal, preferably a chimpanzee. When the cell is present in the body of an animal, the polynucleotide that includes a replication competent HCV can be introduced by, for instance, subcutaneous, intramuscular, intraperitoneal, intravenous, or percutaneous intrahepatic administration, preferably by percutaneous intrahepatic administration. Methods for determining whether an HCV is able to replicate in a chimpanzee are known to the art (see, for example, Yanagi et al., Proc. Natl. Acad Sci. USA, 94, 8738-8743 (1997), and Example 2). In general, the demonstration of infectivity is based on the appearance of the virus in the circulation (blood) of the chimpanzee over the days and weeks following the intrahepatic injection of the HCV. The presence of the virus can be confirmed by reverse transcription-polymerase chain reaction (RT-PCR) detection of the viral RNA, by inoculation of a second chimpanzee with transfer of the hepatitis C virus infection as indicated by the appearance of liver disease and seroconversion to hepatitis C virus in ELISA tests, or possibly by the immunologic detection of components of the hepatitis C virus (e.g., the core protein) in the circulation of the inoculated animal. It should be noted that seroconversion by itself would not be a useful indicator of infection in an animal injected with a viral RNA produced using a molecularly cloned laboratory strain, as this RNA may have immunizing properties and be capable of inducing HCV-specific antibodies to proteins translated from an input RNA that is non-replicating. Similarly, the absence of seroconversion does not exclude the possibility of viral replication and infection of a chimpanzee with HCV.

Whether an HCV is replication competent can be determined using methods known to the art, including methods that use nucleic acid amplification to detect the result of increased levels of HCV replication. Another method for detecting a replication competent HCV includes measuring the production of viral particles by a cell. The measurement of viral particles can be accomplished by passage of supernatant from media containing a cell culture that may contain a replication competent HCV, and using the supernatant to infect a second cell. Detection of HCV in the second cell indicates the initial cell contains a replication competent HCV. The production of infectious virus particles by a cell can also be measured using antibody that specifically binds to an HCV viral particle. As used herein, an antibody that can “specifically bind” an HCV viral particle is an antibody that interacts only with the epitope of the antigen (e.g., the viral particle or a polypeptide that makes up the particle) that induced the synthesis of the antibody, or interacts with a structurally related epitope. “Epitope” refers to the site on an antigen to which specific B cells and/or T cells respond so that antibody is produced. An epitope could includes about 3 amino acids in a spatial conformation which is unique to the epitope. Generally an epitope includes at least about 5 such amino acids, and more usually, consists of at least about 8-10 such amino acids. Antibodies to HCV viral particles can be produced as described herein.

In another aspect, identifying a replication competent HCV includes incubating a cultured cell that includes an HCV having a heterologous polynucleotide, preferably inserted in the 3′ non-translated RNA as described herein. In those aspects of the invention where the heterologous polynucleotide encodes a detectable marker, cells containing a replication competent HCV can be identified by observing individual cells that contain the detectable marker. Alternatively, if the detectable marker is secreted by the cell, the presence of the marker in the medium in which the cell is incubated can be detected. Methods for observing the presence or absence of a detectable marker in a cell or in liquid media are known to the art.

Another, more preferred aspect of the invention allows the positive selection of cells that include a replication competent HCV. The marker expressed by the HCV is a selectable marker, and the cell, which includes the HCV, is incubated in the presence of a selecting agent. Those cells that can replicate in the presence of the selecting agent contain an HCV that is replication competent. Typically, the cells that can replicate are detected by allowing resistant cells to grow in the presence of the selecting agent.

Optionally and preferably, the method further includes isolating virus particles from the cells that contain a replication competent HCV and exposing a second cell to the isolated virus particle under conditions such that the virus particle is introduced to the cell. After providing time for expression of the selectable marker, the second cell is then incubated with the selecting agent. The presence of a cell that replicates indicates the replication competent HCV produces infectious virus particles. Preferably, virus particles are isolated by removing a volume of the media in which the first cells are incubated.

In another aspect, the invention provides a method for detecting a replication competent HCV. The method includes incubating a cell that contains an HCV including a heterologous polynucleotide, preferably inserted in the 3′ non-translated RNA as described herein. The cell includes a transactivated coding region and an operator sequence operably linked to the transactivated coding region. The transactivated coding region encodes a detectable marker.

The heterologous polynucleotide present in the HCV encodes a transactivator that binds to the operator sequence present in the cell. The binding of the transactivator to the operator sequence can decrease transcription or increase transcription of the operably linked transactivated coding region. Preferably, binding of the transactivator to the operator sequence increases transcription. Preferably, the HCV also encodes a marker, more preferably, a fusion polypeptide that includes a transactivator and a marker. Most preferably, the fusion polypeptide further includes a cis-acting proteinase located between the nucleotides encoding the transactivator and the nucleotides encoding the marker.

The method further includes detecting the presence or absence of the detectable marker encoded by the transactivated coding region present in the cell. The presence of the detectable marker indicates the cell comprises a replication competent hepatitis C virus. Preferably, the detectable marker is one that is secreted by the cell, for instance secretory alkaline phosphatase.

The methods described above for identifying replication competent HCV can also be used for identifying a variant HCV, i.e., an HCV that is derived from a replication competent HCV of the present invention. Preferably, a variant HCV has a faster replication rate than the parent or input HCV. The method takes advantage of the inherently high mutation rate of RNA replication. It is expected that during continued culture of a replication competent HCV in cultured cells, the HCV of the present invention will mutate, and some mutations will result in HCV with greater replication rates. The method includes identifying a cell that has greater expression of a polypeptide encoded by a replication competent HCV. An HCV of the present invention that replicates at a faster rate will result in more of the polypeptide(s) that is encoded by the heterologous polynucleotide present in the HCV. For instance, when an HCV encodes a selectable marker, a cell containing a variant HCV having a greater replication rate will be resistant to higher levels of an appropriate selecting agent. When an HCV encodes a transactivator, a cell containing a variant HCV having a greater replication rate than the parent or input HCV will express higher amounts of the transactivated coding region that is present in the cell. The observed increases in resistance to phleomycin D1 (for instance, ZEOCIN) suggest the accumulation of mutations that allow increased rates of replication. Such mutations are referred to herein as “cell culture adaptive mutations.”

Specific cell culture adaptive mutations have been characterized for HCV 1b subgenomic RNA replicons (see, for instance, Blight et al., Science, 290, 1972-1975 (2000); Lohmann et al., “Adaptation of selectable HCV replicon to a human hepatoma cell line,” Abstract P038, 7th International Meeting on Hepatitis C virus and Related viruses (Molecular Virology and Pathogenesis), The Marriott resort Hotel, Gold coast, Queensland, Australia, December 3-7 (2000); and Guo et al., “Identification of a novel RNA species in cell lines expressing HCV subgenomic replicons,” Abstract P045, 7th International Meeting on Hepatitis C virus and Related viruses (Molecular Virology and Pathogenesis), The Marriott resort Hotel, Gold coast, Queensland, Australia, December 3-7 (2000)). It is expected that the introduction of these individual mutations may enhance the replication capacity of an HCV of the present invention. The approximate locations and types of mutations are shown in Table 1. The precise location of these cell culture adaptive mutations can vary between members of different genotypes, and between members of the same genotype. For instance, with mutations 2442 and 2884 listed in Table 1, in HCV genotype 1a the locations of these mutations are 2443 and 2885, respectively. The location of a mutation introduced into an HCV of the present invention to enhance replication is expected to be within 4 amino acids, preferably within 3 amino acids, more preferably within 2 amino acids, most preferably within 1 amino acid of the positions listed in Table 1.

TABLE 1
Adaptive mutations.

| Amino acid position¹ | Mutation² |
|---|---|
| 1283 | R to G |
| 1383 | E to A |
| 1577 | K to R |
| 1609 | K to E |
| 1936 | P to S |
| 2163 | E to G |
| 2330 | K to E |
| 2442 | I to V |
| 2884 | R to G |
| 2177 | D to H, or D to N |
| 2189 | R to G |
| 2196 | P to S |
| 2197 | S to P, or S to C |
| 2199 | A to S, or A to T |
| 2201 | deletion of S |

¹Amino acid position refers to amino acid number where the first amino acid is the first amino acid of the polyprotein expressed by the HCV at Genbank Accession number AJ238799.
²Amino acids are listed in the single letter code. The first amino acid is the wildtype amino acid, and the second amino acid is the residue present in the mutant.

Cell culture adaptive mutations can be introduced into an HCV of the present invention by mutagenesis of the nucleotide sequence of the HCV in the form of plasmid DNA. Methods for targeted mutagenesis of nucleotide sequences are known to the art, and include, for instance, PCR mutagenesis.

A cDNA molecule of a variant HCV can be cloned using methods known to the art (see, for instance, Yanagi et al., Proc. Natl. Acad. Sci., USA, 94, 8738-8743 (1997)). The nucleotide sequence of the cloned cDNA can be determined using methods known to the art, and compared with that of the input RNA. This allows identification of mutations that have occurred in association with passage of the HCV in cell culture. For example, using methods known to the art, including longrange RT-PCR, extended portions of a variant HCV genome can be obtained. Multiple clones could be obtained from each segment of the genome, and the dominant sequence present in the culture determined. Mutations that are identified by this approach can then be reintroduced into the background of the HCV cDNA encoding the parent or input HCV. Preferably, this is used to produce a replication competent HCV that does not contain a heterologous polynucleotide. Such an HCV would have superior replication properties in cell culture compared to the parent HCV and the variant HCV because it would not carry the burden of an additional coding region within its 3′ non-translated RNA.

The present invention also provides methods for identifying a compound that inhibits replication of an HCV, preferably a replication competent HCV as described herein in the section “Hepatitis C Virus.” The method includes contacting a cell containing a replication competent HCV with a compound and incubating the cell under conditions that permit replication of the replication competent HCV in the absence of the compound. After a period of time sufficient to allow replication of the HCV, the presence of replication competent hepatitis C virus is detected. A decrease in the presence of replication competent HCV in the cell contacted with the compound relative to the presence of replication competent HCV in a cell not contacted by the compound indicates the compound inhibits replication of a replication competent HCV. A compound that inhibits replication of an HCV includes compounds that completely prevent replication, as well as compounds that decrease replication. Preferably, a compound inhibits replication of a replication competent HCV by at least about 50%, more preferably at least about 75%, most preferably at least about 95%.

The compounds added to a cell can be a wide range of molecules and is not a limiting aspect of the invention. Compounds include, for instance, a polyketide, a non-ribosomal peptide, a polypeptide, a polynucleotide (for instance an antisense oligonucleotide or ribozyme), or other organic molecules. The sources for compounds to be screened include, for example, chemical compound libraries, fermentation media of Streptomycetes, other bacteria and fungi, and extracts of eukaryotic or prokaryotic cells. When the compound is added to the cell is also not a limiting aspect of the invention. For instance, the compound can be added to a cell that contains a replication competent HCV. Alternatively, the compound can be added to a cell before or at the same time that the replication competent HCV is introduced to the cell.

Typically, the ability of a compound to inhibit replication of a replication competent virus is measured using methods described herein. For instance, methods that use nucleic acid amplification to detect the amount of HCV nucleic acid in a cell can be used. Alternatively, methods that detect or select for a marker encoded by a replication competent HCV or encoded by a cell containing a replication competent HCV can be used.

The replication competent HCV of the invention can be used to produce infectious viral particles. For instance, a cell that includes a replication competent HCV can be incubated under conditions that allow the HCV to replicate, and the infectious viral particles that are produced can be isolated, preferably purified. The infectious viral particles can be used as a source of virus particles for various assays, including evaluating methods for inactivating particles, excluding particles from serum, identifing a neutralizing compound, and as an antigen for use in detecting anti-HCV antibodies in an animal. An example of using a viral particle as an antigen includes use as a positive-control in assays that test for the presence of anti-HCV antibodies.

For instance, the activity of compounds that neutralize or inactivate the particles can be evaluated by measuring the ability of the molecule to prevent the particles from infecting cells growing in culture or in cells in an animal. Inactivating compounds include detergents and solvents that solubilize the envelope of a viral particle. Inactivating compounds are often used in the production of blood products and cell-free blood products. Examples of compounds that can be neutralizing include a polyketide, a non-ribosomal peptide, a polypeptide (for instance, an antibody), a polynucleotide (for instance, an antisense oligonucleotide or ribozyme), or other organic molecules. Preferably, a neutralizing compound is an antibody, including polyclonal and monoclonal antibodies, as well as variations thereof including, for instance, single chain antibodies and Fab fragments.

Viral particles produced by replication competent HCV of the invention can be used to produce antibodies. Laboratory methods for producing polyclonal and monoclonal antibodies are known in the art (see, for instance, Harlow E. et al. Antibodies: A laboratory manual Cold Spring Harbor Laboratory Press, Cold Spring Harbor (1988) and Ausubel, R. M., ed. Current Protocols in Molecular Biology (1994)), and include, for instance, immunizing an animal with a virus particle. Antibodies produced using the viral particles of the invention can be used to detect the presence of viral particles in biological samples. For instance, the presence of viral particles in blood products and cell-free blood products can be determined using the antibodies.

The present invention further includes methods of treating an animal including administering neutralizing antibodies. The antibodies can be used to prevent infection (prophylactically) or to treat infection (therapeutically), and optionally can be used in conjunction with other molecules used to prevent or treat infection. The neutralizing antibodies can be mixed with pharmaceutically acceptable excipients or carriers. Suitable excipients include but are not limited to water, saline, dextrose, glycerol, ethanol, or the like and combinations thereof. In addition, if desired, neutralizing antibodies and pharmaceutically acceptable excipients or carriers may contain minor amounts of auxiliary substances such as wetting or emulsifying agents, pH buffering agents, and/or adjuvants which enhance the effectiveness of the neutralizing antibodies. Such additional formulations and modes of administration as are known in the art may also be used.

The virus particles produced by replication competent HCV of the invention can be used as a source of viral antigen to measure the presence and amount of antibody present in an animal. Assays are available that measure the presence in an animal of antibody directed to HCV, and include, for instance, ELISA assays, and recombinant immunoblot assay. These types of assays can be used to detect whether an animal has been exposed to HCV, and/or whether the animal may have an active HCV infection. However, these assays do not use virus particles, but rather individual or multiple viral polypeptides expressed from recombinant cDNA that are not in the form of virus particles. Hence they are unable to detect potentially important antibodies directed against surface epitopes of the envelope polypeptides, nor are they measures of functionally important viral neutralizing antibodies. Such antibodies could only be detected with the use of infectious virus particles, such as those that are produced in this system. The use of infectious viral particles as antigen in assays that detect the presence of specific antibodies by virtue of their ability to block the infection of cells with HCV viral particles, or that possibly bind to whole virus particles in an ELISA assay or radioimmunoassay, will allow the detection of functionally important viral neutralizing antibodies

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Construction of the Infectious MK0-Z RNA

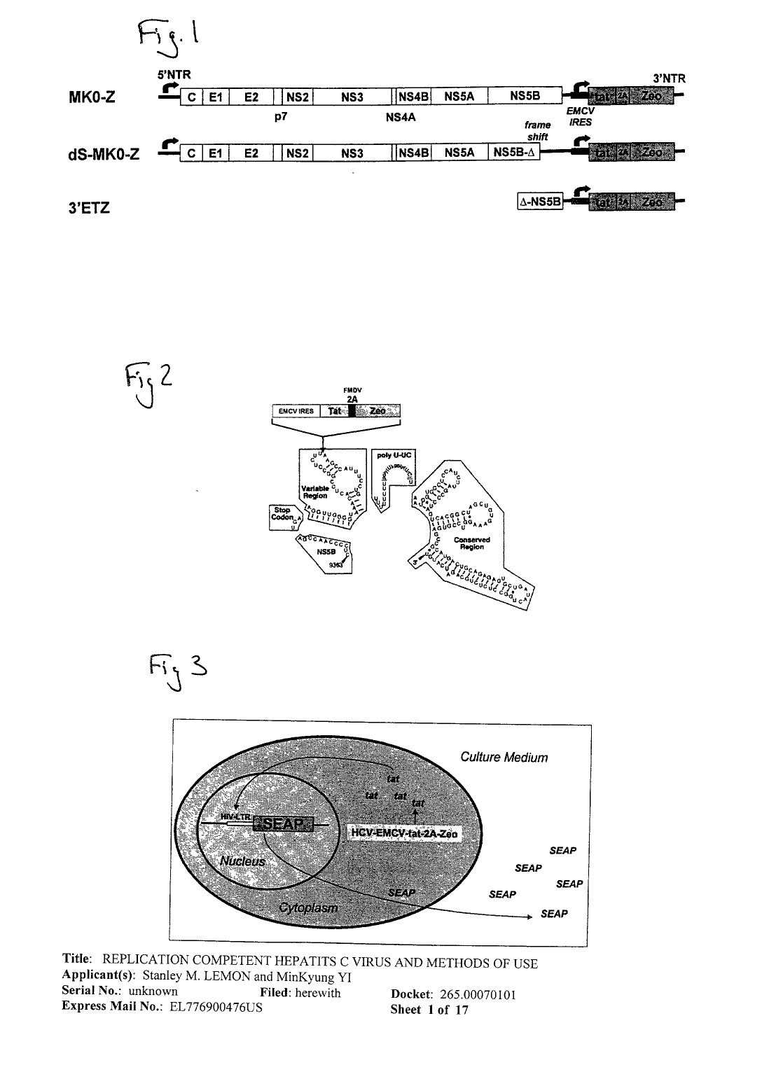

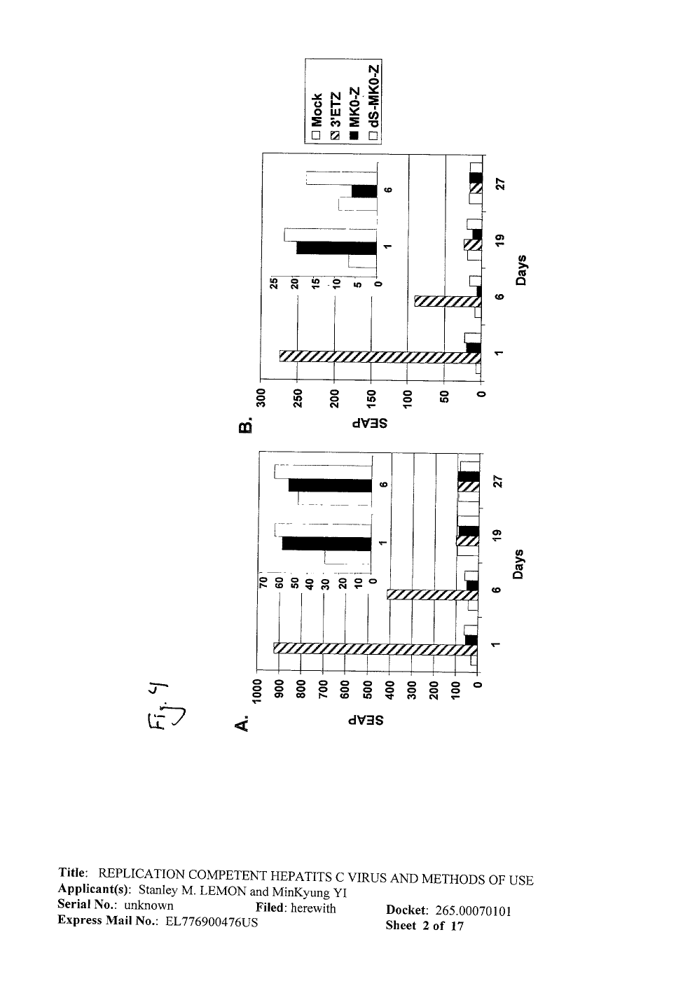

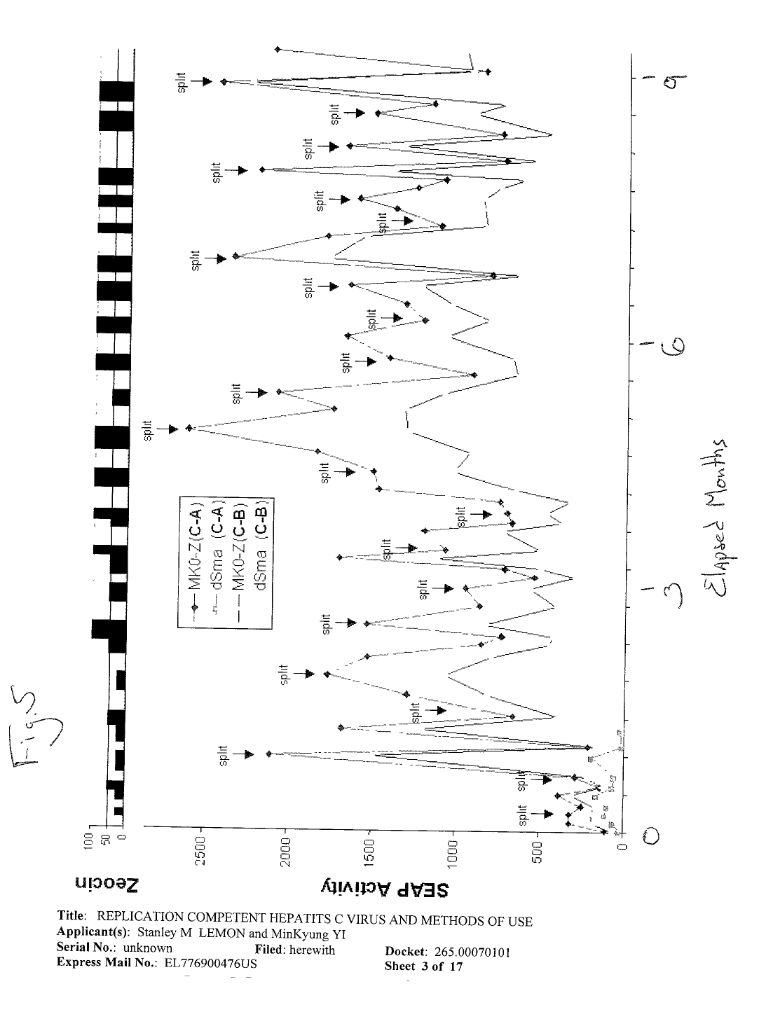

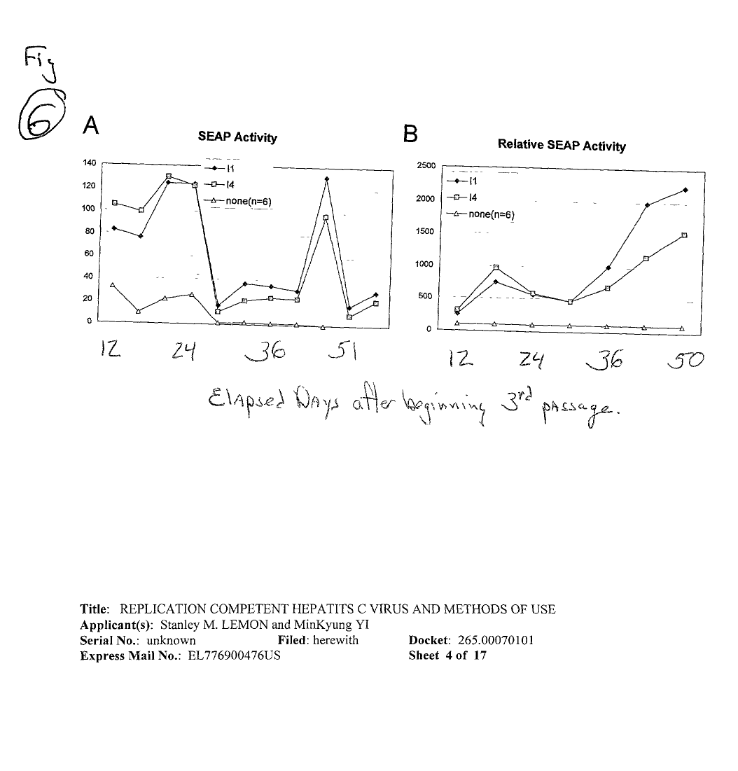

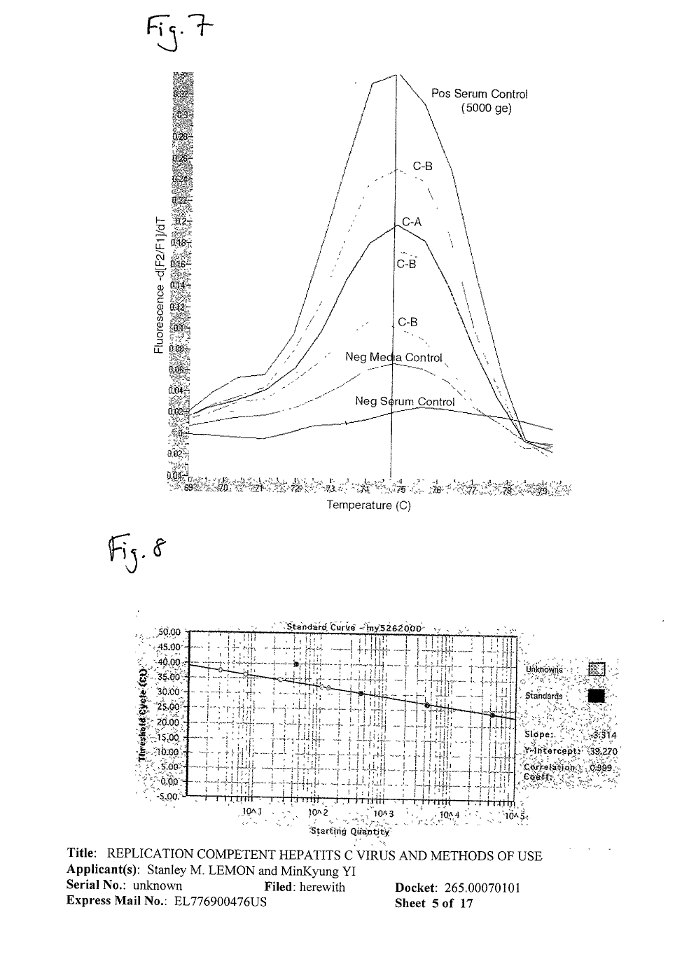

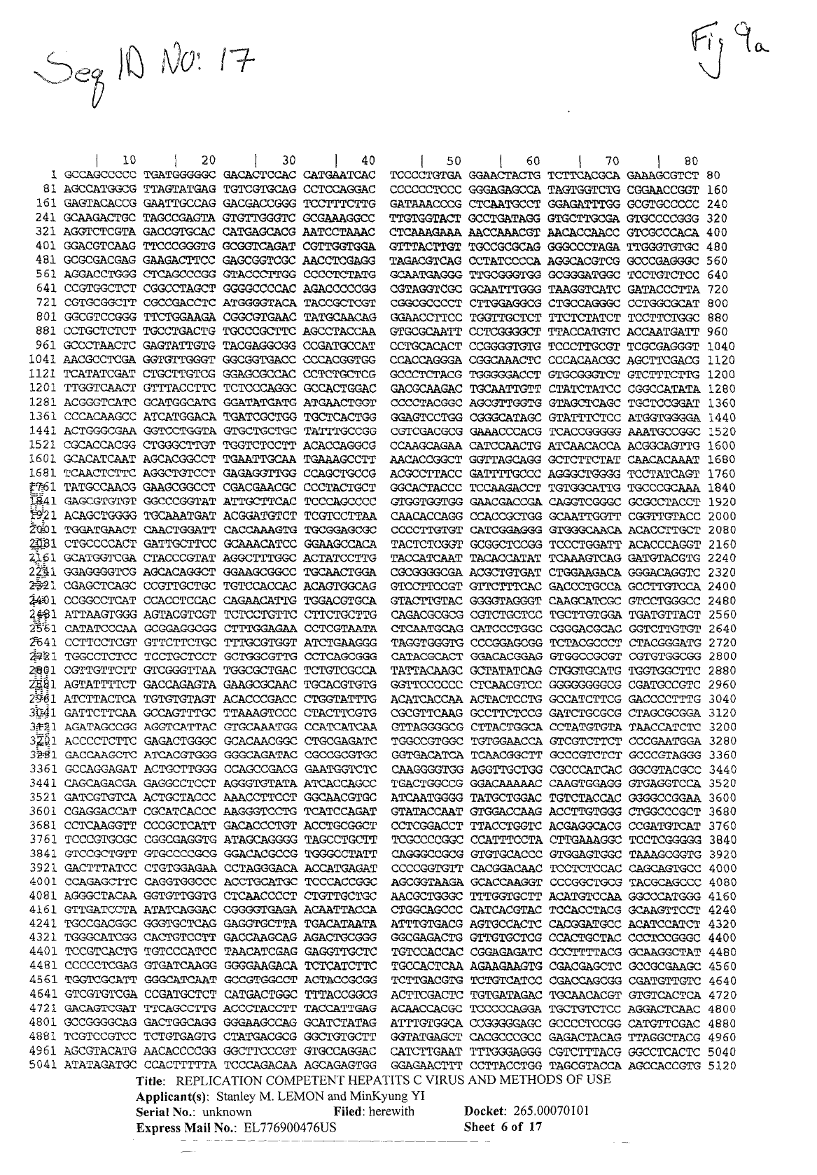

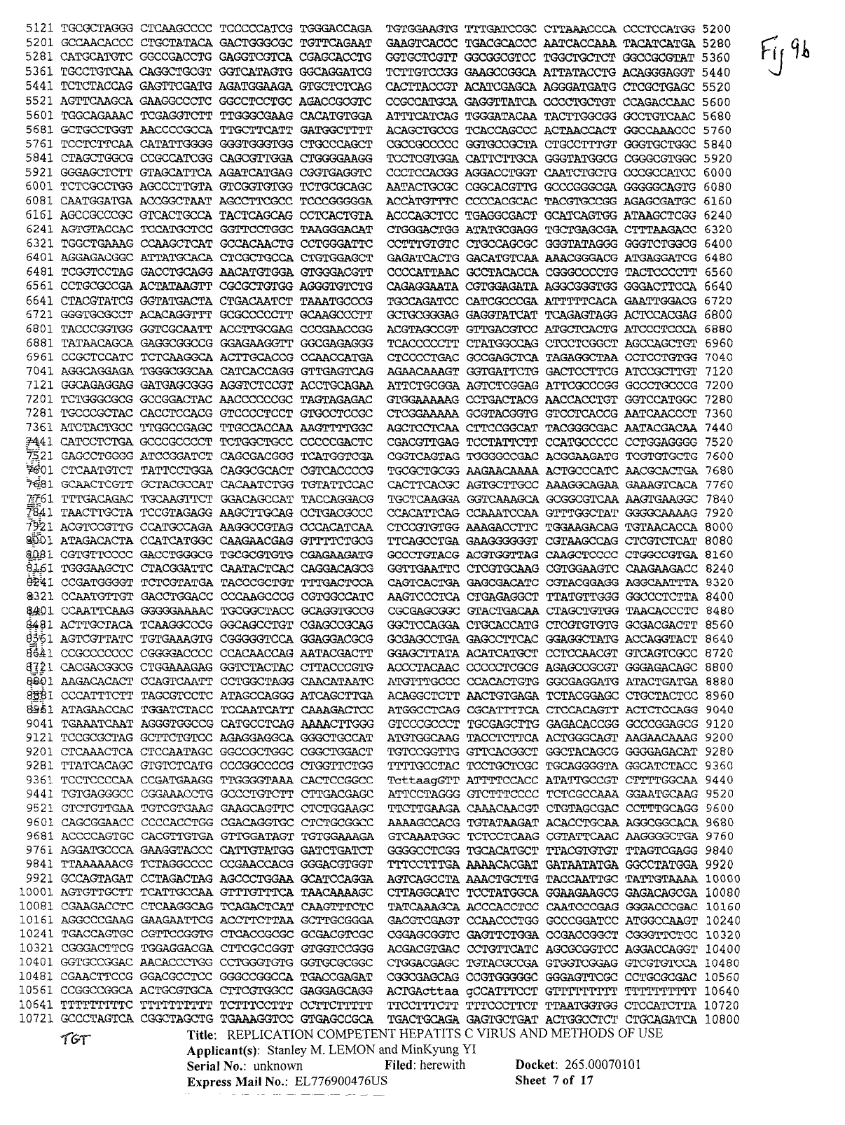

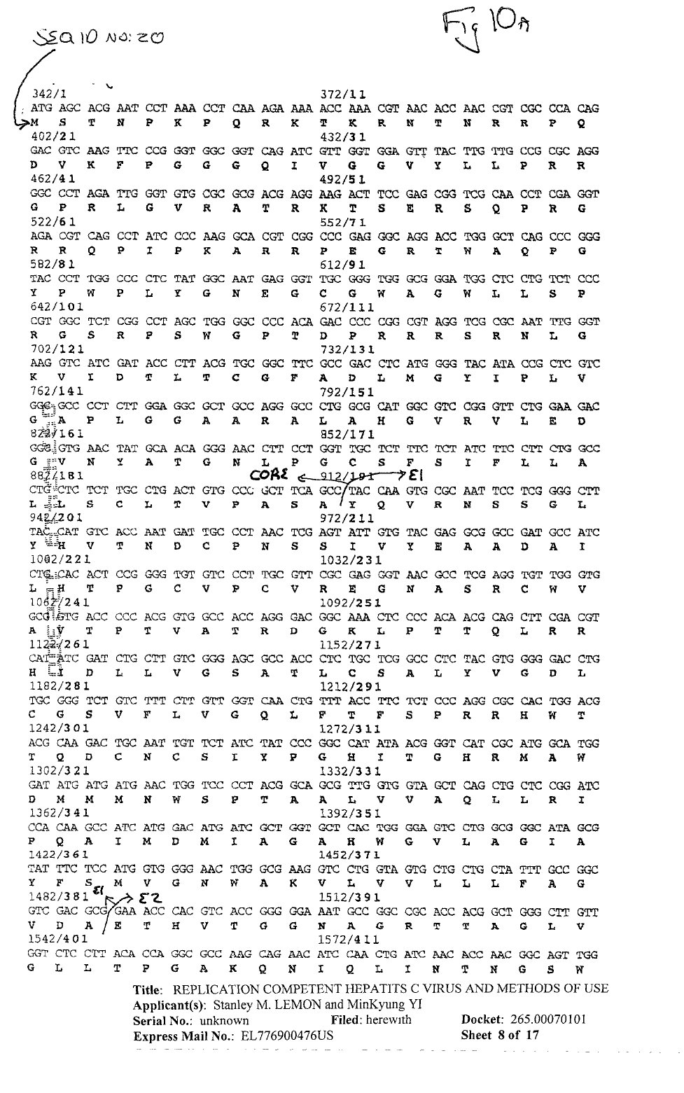

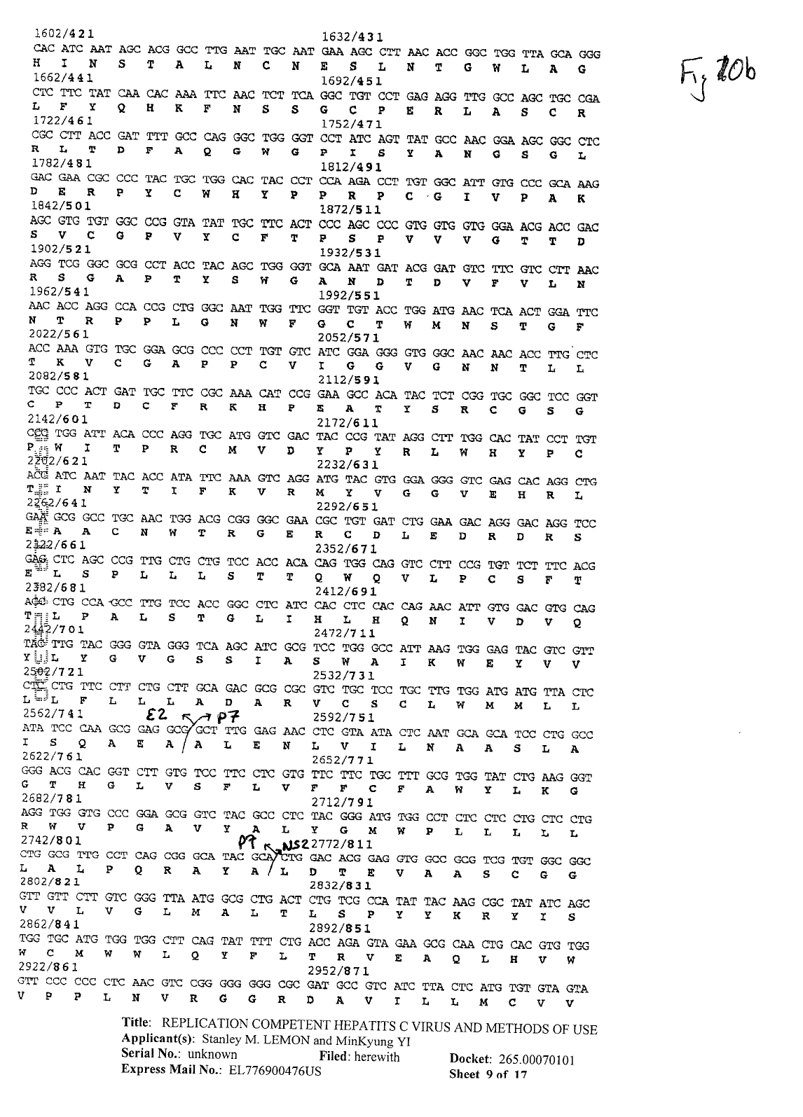

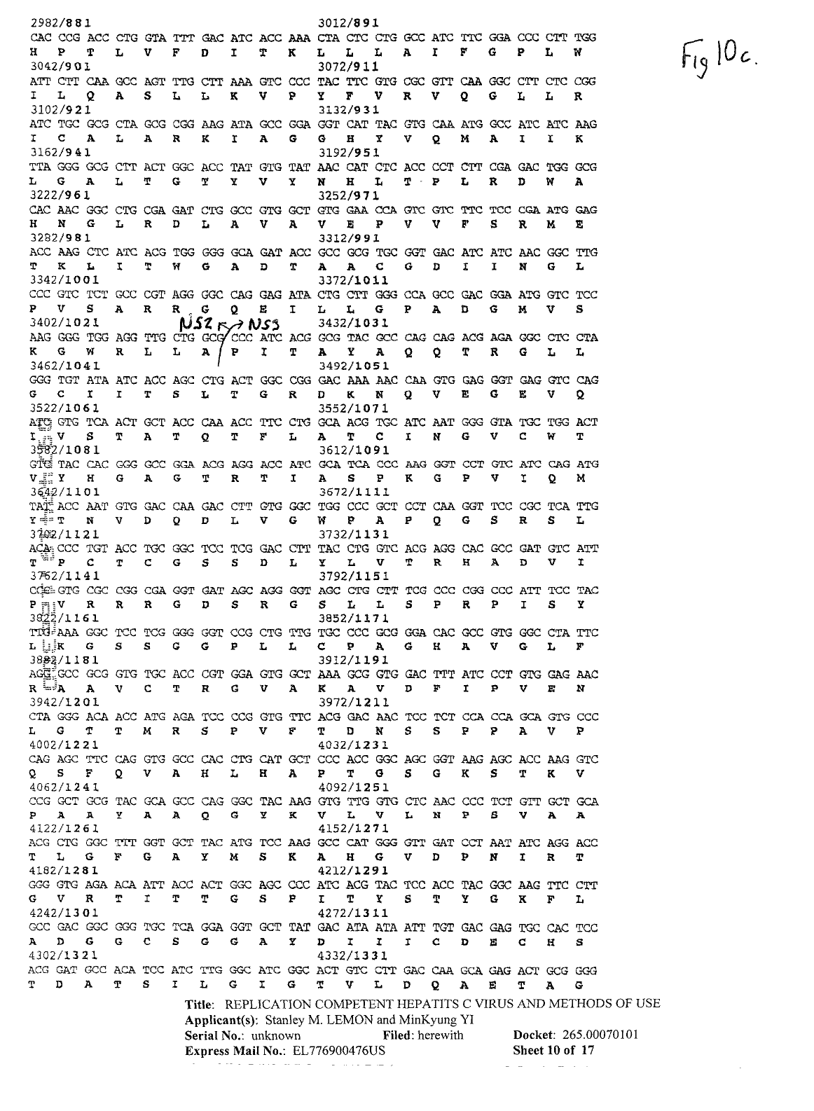

FIG. 1 shows the full-length modified HCV cDNA (MK0-Z) that was constructed by modification of pCV-H77C. The nucleotide sequence of MK0-Z is shown in FIG. 9. A coding region encoding a polypeptide conferring resistance to neomycin has been expressed under control of the EMCV IRES from a second reading frame inserted within the 3′ non-translated RNA in subgenomic Kunjin virus replicons. However, the specific placement of the foreign sequence could not be used as a guide for the placement of a coding region in HCV since the 3′ non-translated RNA of these viruses share no sequence identity. In the case of MK0-Z, the heterologous sequence functions as a unique 3′ cistron, with the internal ribosome entry site (IRES) of encephalomyocarditis virus (EMCV) directing the cap independent translation of a novel polyprotein composed of Tat and the ZEOCIN (phleomycin, Invitrogen) resistance protein, Zeo, separated by the cis-active 2A proteinase of foot-and-mouth disease (FMDV) virus. The Asn-Pro-Gly sequence at the carboxy terminus of FMDV 2A mediates proteolytic cleavage at the 2AZeo junction, effectively separating the upstream Tat and downstream Zeo polypeptides (Ryan et al., EMBO J., 13, 928-933 (1994)). The heterologous sequence is placed within the 3′NTR of HCV, a genomic region that contains highly conserved sequences that cannot be deleted without loss of infectivity. More specifically, the heterologous sequence was placed within the variable region of the 3′NTR (FIG. 2). As a control, a replication-incompetent variant of MK0-Z, dS-MK0-Z, was constructed by opening the clone at two closely positioned Sma I sites within the NS5B coding region, then religating the plasmid. This resulted in a frame-shift deletion in the HCV sequence, upstream of the GDD motif in the polymerase encoded by the NS5B coding region, that is lethal to viral replication. The novel 3′ reading frame in MK0-Z, has been shown to be active translationally in in vitro translation reactions carried out in rabbit reticulocyte lysates. These experiments also demonstrated that the 2A proteinase effectively cleaved the resulting polyprotein, releasing Tat-2A from the Zeo protein.

a. Construction of pUC HCV3′-EMCV-tat-2A-Zeo

To make pHCV3′, full length HCV 1a (present on the plasmid pCV-H77C) (provided by Dr. Purcell at NIH) was digested with HindIII-XbaI. A DNA fragment of about 1.7 kilobases, corresponding to nucleotides 7861- 9599 of the HCV nucleotide sequence available at Genbank Accession number AF011751, was isolated and ligated into the vector pBluescript (Stratagene) that had been digested with HindIII and XbaI. The resulting plasmid was designated pHCV3′.

A DNA fragment containing the EMCV IRES was generated by the polymerase chain reaction (PCR). The plasmid pEMCV-CAT, described in Whetter et al., (Arch. Virol., Suppl. 9, 291-298 (1994)) was amplified using the sense primer 5′-GGCCTCTTAAGGTTATTTTCCACCATATTGCC (SEQ ID NO:22) which contained a BfrI site, and the anti-sense primer 5′-TCCCCGCGGAAGGCCTCATATTATCATCGTGTTTTTC (SEQ ID NO:23) which contained a SacI and StuI site. The italicized nucleotides are those which are not present in the DNA to be amplified, and the underlined nucleotides indicate a restriction endonuclease site. The PCR conditions were: 94° C. for 30 seconds, 55° C. for 30 seconds, and 72° C. for 1 minute, for 35 cycles.

pHCV3′-EMCV was generated by ligating EMCV IRES fragment digested with BfrI-SacI and vector from pHCV3′ digested with same enzymes.

A DNA fragment containing the nucleotides encoding 85 amino acids from the HIV I Tat protein was generated by PCR. The amino acid sequence of the HIV I Tat protein is shown at amino acids 4-89 of SEQ ID NO:21 The plasmid used was pCTAT (provided by Dr. Bryan Cullen, Duke University, Durham, N.C. Dept. of Microbiology) (see Bieniasz et al., Molecular Cellular Biology, 19 4592-4599); was amplified using the sense primer 5′-GAAGGCCTATGGAGCCAGTAGATCCTAGA (SEQ ID NO:28), which contained a StuI site, and and anti-sense primer 5′-CGGAATCTTCCTTCGGGCCTGTCGGGTCC (SEQ ID NO:29), which contained an EcoRI site. The italicized nucleotides are those which are not present in the DNA to be amplified, and the underlined nucleotides indicate a restriction endonuclease site. The PCR conditions were: 94° C. for 30 seconds, 55° C. for 30 seconds, and 72° C. for 1 minute, for 35 cycles.

A DNA fragment containing the nucleotides encoding 15 amino acids of FMDV 2A was generated by annealing 51 mer primer set; sense primer 5′-

| | |
|---|---|
| AATTCGACCTTCTTAAGCTTGCGGGAGACGTCGAGTCCAACCCTGGGCCCG | (SEQ ID NO:24) |
| and anti-sense primer 5′- | |
| GATCCGGGCCCAGGGTTGGACTCGACGTCTCCCGCAAGCTTAAGAAGGT | |

CG (SEQ ID NO:25) with putative digested form of EcoRI and BamHI site at its 5′ and 3′ end, respectively. The result was a DNA fragment encoding the 15 amino acids of FMDV 2A. The amino acid sequence encoded by the DNA fragment was FDLLKLAGDVESNPG (SEQ ID NO:30).

A DNA fragment containing the coding region encoding resistance to phleomycin was generated by the polymerase chain reaction (PCR). The plasmid pZeoSV (Invitrogen) was amplified using the sense primer 5′-CCGCTCGAGGCCTGGATCCATGGCCAAGTTGACCAGTGCC (SEQ ID NO:26) which contained a BamHI site, and anti-sense primer 5′-GGCCTCTTAAGTCAGTCCTGCTCCTCGGCCACG (SEQ ID NO:27) which contained a BfrI site. The italicized nucleotides are those which are not present in the DNA to be amplified, and the underlined nucleotides indicate a restriction endonuclease site. The PCR conditions were: 94° C. for 30 seconds, 55° C. for 30 seconds, and 72° C. for 1 minute, for 35 cycles.

pΔHCV3′-2A-Zeo was generated by digesting the DNA fragment containing the coding region encoding resistance to phleomycin with BfrI-BamHI, and pHCV3′ was with EcoRI-BfrI. These two fragments and the FMDV 2A fragment (which contains an EcoRI site with staggered ends and a BamH site with staggered ends) were then ligated to form pΔHCV3′-2A-Zeo.

pUC HCV3′-EMCV-tat-2A-Zeo was generated by ligating 4 fragments together. A DNA fragment containing the EMCV IRES was obtained by digesting pHCV3′-EMCV with SphI-StuI. The amplified DNA fragment encoding a portion of the HIV I Tat protein was digested with StuI-EcoRI. pΔHCV3′-2A-Zeo was digested with EcoRI and XbaI to yield a DNA fragment containing the nucleotides encoding the FMVD 2A and phleomycin resistance. pUC20 vector digested with SphI-XbaI. These were ligated together and the resulting plasmid was designated pUC HCV3′-EMCV-tat-2A-Zeo.

b. Construction of pUC HCV3′-EMCV-tat-2A Containing New HCV3′ Fragment

Original full length HCV 1a(present on the plasmid pCV-H77C) was digested with SphI-BfrI and a 342 nucleotide fragment (corresponding to nucleotides 9060-9427 of HCV) was isolated. pUC HCV3′-EMCV-tat-2A-Zeo was digested StuI-BamHI and a fragment of 317 nucleotides containing tat-2A was isolated. The remaining portion of the plasmid was digested with BfrI, and a 508 nucleotide BfrI-StuI fragment containing the EMCV IRES was isolated. The remaining 361 nucleotide fragment, which contained the nucleotides encoding phleomycin resistance was isolated and reserved for later use in the construction of pUC Zeo-HCV3′NTR containing new HCV3′NTR fragment (see section c below).

pUC HCV3′-EMCV-tat-2A was generated by ligating the 3 fragments described above, i.e., the 342 nucleotide SphI-BfrI fragment corresponding to nucleotides 9060-9427 of HCV, the 508 nucleotide BfrI-StuI fragment containing the EMCV IRES, and the 317 nucleotide StuI-BamHI fragment containing tat-2A, with the vector pUC20 that had been digested with SphI-BamHI. The resulting plasmid was designated pUC HCV3′-EMCV-tat-2A.

c. Construction of pUC Zeo-HCV3′NTR Containing New HCV3′NTR Fragment

pUC Zeo-HCV3′NTR was constructed by ligating the 361 nucleotide BamHI-BfrI fragment encoding phleomycin resistance (see above), a 198 nucleotide fragment (corresponding to nucleotides 9427-9625 of HCV) generated by digesting original full length HCV 1a with BfrI-XbaI, and the vector pUC20 that had been digested with BamHI-XbaI.

d. Construction of MK0-Z RNA

Steps b and c above were repeated to produce a second pUC HCV3′-EMCV-tat-2A and a second pUC Zeo-HCV3′NTR containing new HCV3′NTR fragment for use in the construction of MK0-Z RNA.

MK0-Z was generated by the ligation of 4 fragments. Full length HCV was digested with HindIII-SphI and a 1,199 nucleotide fragment (corresponding to nucleotides 7861-9060 of HCV) was isolated. A SphI-BamHI DNA fragment containing HCV3′-EMCV-tat-2A was isolated from pUC HCV3′-EMCV-tat-2A. A BamHI-XbaI DNA fragment containing Zeo-HCV3′NTR was isolated from pUC Zeo-HCV3′NTR. Nucleotides corresponding to nucleotides 1-7860 were isolated from pCV-H77C by digestion with HindIII-XbaI. Ligation of these 4 fragments resulted in MK0-Z.

e. Construction of ds-MK0-Z RNA

The plasmid pHCV3′ was digested with SmaI and ligated under conditions to result in self-ligation. The result of the self ligation was loss of the nucleotides corresponding to nucleotides 8497-8649 of HCV. The resulting plasmid was designated pds-HCV3′.

ds-MK0-Z was generated by ligation of 4 DNA fragments. pds-HCV3′ was digested with HindIII-SphI to yield a DNA fragment corresponding to nucleotides 7861-9060 of HCV and containing the SmaI fragment deletion. pUC HCV3′-EMCV-tat-2A was digested with SphI-BamHI to yield a fragment containing HCV3′-EMCV-tat-2A. pUC Zeo-HCV3′NTR was digested with BamHI-XbaI to yield a fragment containing the nucleotides encoding Zeo-HCV3′NTR. Nucleotides corresponding to nucleotides 1-7860 were isolated from pCV-H77C by digestion with HindIII-XbaI. Ligation of these 4 fragments resulted in ds-MK0-Z.

### Example 2

### Production of the Virus by Chimpanzee

This demonstrates the insertion of a heterologous sequence into an HCV does not destroy the ability of the HCV to replicate and produce infectious virus.

MK0-Z plasmid was linearized with XbaI and RNA was synthesized with T7 mega transcription kit from Ambion. The reaction was analysed by gel electrophoresis before injecting into the liver of an HCV-naive Chimpanzee. RNA was frozen at −70° C. overnight before used. About 300 μg of RNA was injected. When injecting, the RNA, which was in 100 ml of transcription reaction mixture, was diluted in 1 ml PBS. The RNA was administered to a Chimpanzee by percutaneous intrahepatic injection guided by ultrasound. Several sites and injections were done in single day. The levels of ALT in the chimpanzee were monitored and were in normal ranges throughout the experiment. Sera from the chimpanzee were collected weekly, and the presence of HCV in each 1 ml of those sera, were checked by RT-PCR, using either the TaqMan or Light Cycler RT-PCR methods.

The primers and probe used for the TaqMan RT-PCR were sense primer, AAGACTGCTAGCCGAGTAGTGTT nt 243 to 265 (SEQ ID NO:1); anti-sense primer: GGTTGGTGTTACGTTTGGTTT nt 390 to 370 (SEQ ID NO:2); and probe: TGCACCATGAGCACGAATCCTAAA nt 336 to 359 (SEQ ID NO:3), where “nt 243 to 265,” “nt 390 to 370,” and “nt 336 to 359” refers to the HCV nucleotides (at Genbank Accession number AF011751) to which the primers hybridize. All single-tube EZ RT-PCR reactions were carried out in optical MicroAmp reaction tubes with optical lids in 50 microliter (μl) volume (96 well format). The RNA amplification was done using the TaqMan EZ RT-PCR Kit. Briefly, reactions contained 1×amplification buffer (TaqMan EZ Buffer), 3 mM manganese, 0.5 U AmpErase uracil-N-glycosylate, 7.5 U rTth DNA polymerase, RNA, 200 nM forward and reverse primers, 200 μM each dNTP, and 500 uM of dUTP. Thermocycling conditions were one cycle at 50° C. for 2 minutes, one cycle at 60° C. for 30 minutes, one cycle at 95° C. for 5 minutes, and 40 cycles of 95° C. for 20 seconds, 60° C. for 1 minute. Amplifications were evaluated by ABI7700 Sequence Detector version 1.6.3 software (Applied Biosystems), as suggested by the manufacturer.

The primers and probe used for Light Cycler RT-PCR were forward primer, ACACTCCACCATGAATCACTC, nt 22 to 41, (SEQ ID NO:4); reverse primer, GATCGGGCTCATCACAACCC, nt 268 to 250, (SEQ ID NO:5); fluor probe, GCGTCTAGCCATGGCGTTAGTATGAGT(fluor), nt 75 to 101 (SEQ ID NO:6); and red probe, (LC640) TCGTGCAGCCTCCAGGACCCC(phosphate), nt 103 to 123 (SEQ ID NO:7). The terms “nt 22 to 41,” “nt 268 to 250,” “nt 75 to 101” and “nt 103 to 123” refer to the HCV nucleotides (at Genbank Accession number AF011751) to which the primers hybridize. The “fluor probe” is labeled at the 3′ end with fluorescein, and the “red probe” is labeled at the 5′ with LightCycler Red 640 dye.

Single-tube RT-PCR reactions were carried out in capillary tubes in a reaction volume of 20 μl using the core reagents of RNA Amplification Kit Hybridization Probes (Roche) as suggested by the manufacturer. A master mix was made according to the manufacturer's suggestions, containing Lightcycler-RT-PCR Reaction Mix Hybridization probe solution, LightCycler RT-PCR Enzyme mix, 7 mM MgCl₂, 0.5 μM of forward primer, 0.9 μM of reverse primer and 0.5 μM of flour probe, 0.9 μM of red probe, and H₂O is added to make it total 20 μl. This master mix was added directly to the RNA pellet and after dissolve the RNA, it was loaded into glass capillary tube. After adding the 5 ul wash, the tube was snap sealed with a plastic cap. The RT-PCR conditions were 55° C. for 15 minutes, 95° C. for 30 seconds, and 40 cycles of 94° C. for 0 seconds, 60° C. annealing for 15 seconds, and 72° C. extension for 15 seconds.

The signal acquisition was at the end of the annealing step for 100 milliseconds (ms). After amplification was complete, a melting curve was performed by cooling to 55° C., holding at 55° C. for 30 seconds, and then heating slowly at the rate of 0.2 C./second until 90° C. Signal was collected continuously during this melting to monitor the dissociation of the 5′-LC640-labeled probe. The signal was the result of fluorescence resonance energy transfer (FRET) between the fluor probe and the red probe. These probes hybridize to an internal sequence of the amplified fragment during the annealing phase of the PCR cycle. One probe is labeled at the 5′ end with a LightCycler-Red fluorophore (LC-Red 640 or LC-Red 705), and to avoid extension, modified at the 3′ end by phosphorylation. The other probe is labeled at the 3′ end with fluorescein. Only after hybridization to the template, do the two probes come in close proximity, resulting in FRET between the two fluorophores. During FRET, fluorescein, the donor fluorophore, is excited by the light source of the LightCycler Instrument. Part of the excitation energy is transferred to LightCycler-Red, the acceptor fluorophore. The emitted fluorescence of the LightCycler-Red fluorophore is measured. The melting curves were then displayed as—dF/d T vs T plots as calculated by LightCycler software version 3.

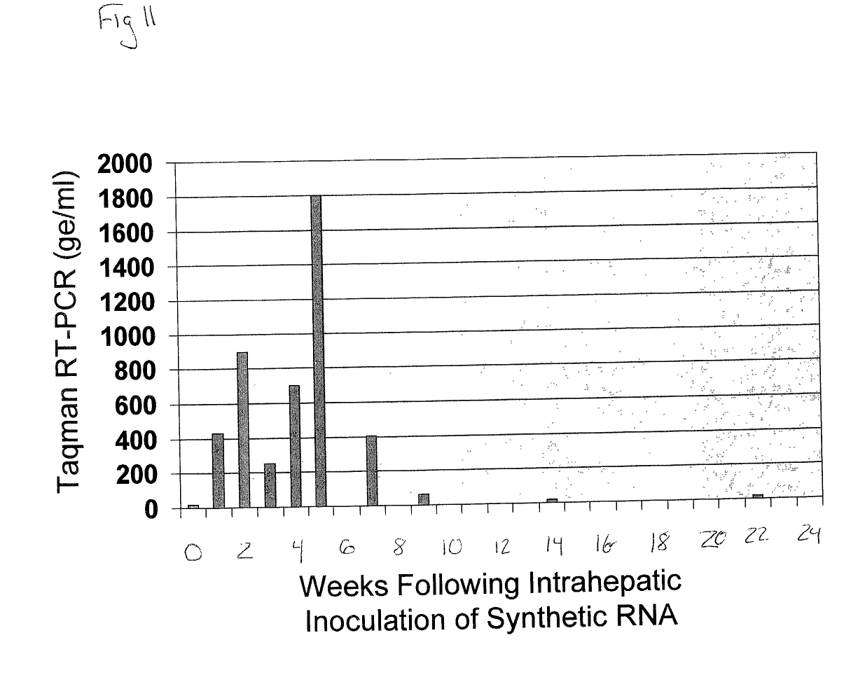

The results of TaqMan RT-PCR are shown in FIG. 11. They demonstrate that MK0-Z RNA is infectious in a chimpanzee.

### Example 3

### Construction of a Cellular Enzyme Reporter System for Detection of Replicating HCV

A major difficulty in evaluating the outcome of experiments in which cultured cells are transfected with candidate infectious RNAs lies in the detection of newly synthesized viral RNAs against the large background of transfected input RNA. While this is less of a problem with very robustly replicating viral RNAs, only Lohmann et al. (Science, 285,110-113 (1999)) and Blight et al. (Science, 290, 1972-1975 (2000)) have thus far reported levels of replication detectable by northern analysis, using subgenomic RNA replicons that are not capable of producing infectious virus. Moreover, these authors observed such replication only in a small number of cell clones that were isolated over a period of weeks by a stringent antibiotic selection protocol. RT-PCR is difficult to use to detect newly replicated nucleic acid in recently transfected cells due to the persistence of input RNA (in our experience, RNA transfected by liposome-mediated methods remains detectable for weeks). The use of a negative-strand “specific” assay reduces, but does not eliminate this problem, since such assays have no more than a −1,000-fold relative specificity for detection of the negative strand vs. detection of the positive-strand (see, for instance, Lanford et al., J. Virol, 69, 8079-8083 (1995)).

This Example details the construction of a cell line that allows the detection of replicating synthetic HCV RNA. The detection is based on the detection of a protein product expressed from the RNA. The system uses the incorporation of the sequence encoding the HIV I Tat protein within modified viral RNAs (see FIG. 1). The Tat protein is a strong transactivator of the HIV I long terminal repeat (LTR) transcriptional regulator. For use as cell substrates in this system, multiple stably transformed cell lines were established. The transformed cell lines were derived from Huh-7 cells that express secretory alkaline phosphatase (SEAP) under transcriptional control of the HIV I LTR. These cell lines were established using either Neomycin or Blastocidin selection, so that either of these antibiotics or Zeocin can be used for subsequent selection of replicating full-length HCV RNAs. The expression of Tat within these cells leads to measurable increases in SEAP activity within the culture medium, as depicted schematically in FIG. 3.

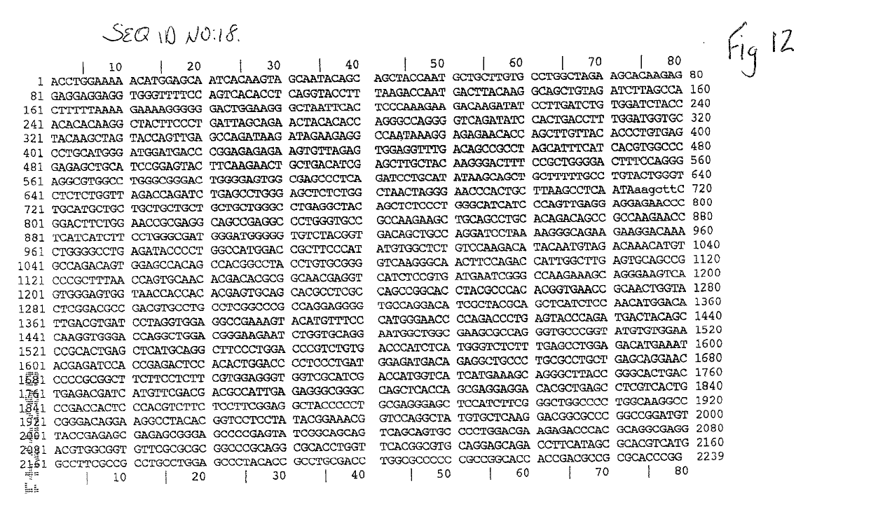

For establishment of neomycin resistant SEAP cell lines, the HIV-SEAP sequence was PCR amplified from pBCHIVSEAP plasmid (provided by Dr. Bryan Cullen, Duke University, Durham, N.C. Dept. of Microbiology) (see Cullen, Cell, 46, 973-982 (1986), and Berger et al., Gene, 66, 1-10 (1988)) using the primer pairs 5′-CTAGCTAGCCTCGAGACCTGGAAAAACATGGAG (SEQ ID NO:8) and 5′-ATAAGAATGCGGCCGCTTAACCCGGGTGCGCGG (SEQ ID NO:9). The non-italicized nucleotides in SEQ ID NOs:8 and 9 hybridize with nucleotides present in the target DNA, and the italicized nucleotides in SEQ ID NO:9 represent additional nucleotides that do not hybridize with the target DNA. The underlined nucleotides indicate introduced restriction endonuclease sites. The nucleotide sequence of the amplified fragment is shown in FIG. 12 (SEQ ID NO:18).

After filling in to repair the possible PCR overhang, this fragment was digested with NotI and ligated to vector derived from pRcCMV (Invitrogen) digested with NruI-NotI removing CMV promoter. The resulting plasmid was designated pRcHIVSEAP The nucleotide sequence of the

pRcHIVSEAP was used to transfect Huh-7 cells using a non-liposomal transfection reagent commercially available under the trade name FUGENE (Boerhinger Manheim). Tranfectants were selected using G418 (neomycin). The ability of a cell to express SEAP in the presence of tat was tested by transfecting cells with the plasmid pCTAT, which expresses the tat protein. Two resulting cell lines which expressed high levels of SEAP were designated Huh-o10 (also referred to as Huh7-SEAP-o10) and Huh7-SEAP-N7, and were used for subsequent experiments.

A Blasticidin resistant SEAP cell line was constructed as follows. pcDNA6/V5-His (Invitrogen) was digested with BglII-BamHI to remove the CMV promoter. The vector was then self-ligated and subsequently digested with EcoRV-NotI and ligated to the HIV-SEAP DNA fragment that was PCR amplified from pBCHIVSEAP fragment mentioned. The resulting plasmid was used to transfect Huh-7 cells using a non-liposomal transfection reagent commercially available under the trade name FUGENE (Boerhinger Manheim). Tranfectants were selected using Blastocidin (Invitrogen). A blastocidin resistant cell was selected and designated Huh-SEAP-Bla-EN.

### Example 4

### Evaluation of the Cellular Enzyme Reporter System for Detection of Replicating HCV

This Example demonstrates the feasibility and utility of the SEAP cellular reporter system, and demonstrates the expression of Tat by the genetically modified HCV RNA.

To test the SEAP cellular reporter system, MK0-Z RNA was synthesized and transfected into two different SEAP reporter cell lines, Huh7-SEAP-o 10 and Huh7-SEAP-N7 (another cell line that resulted from neomycin selection), on the same day. To provide adequate controls for this experiment, cells from both cell lines were transfected with RNAs synthesized from each of the plasmid DNAs shown in FIG. 1. These include MK0-Z, its replication incompetent control dS-MK0-Z, and a subgenomic transcript, 3′ETZ, each of which encode the novel polyprotein consisting of Tat and Zeo separated by the 19 amino acid 2A proteinase from FMDV 4. Fifteen of the amino acids were the FMDV 2A sequence, and 4 additional amino acids were encoded by nucleotides present to introduce restriction endonuclease sites. In each of the transfected RNAs, this polyprotein is under the translational control of the EMCV IRES.

DNA was linearized with Xba I and RNA was synthesized with T7 mega transcription kit (Ambion, Madison, Wis.). Transfection of RNA was done using Lipofectin (Gibco BRL, Rockville, Md.). Briefly, about 5 μg of RNA was added to a mixture (1 hour incubation prior to transfection) of 15 μl of Lipofectin and 200 μl OPTIMEM (Gibco BRL), incubated for 15 min, and applied to cells. The cells were in 6 well plates which had been plated one day before transfection. The cells were washed two times with OPTIMEM before addition of the RNA, followed by the addition of 1 ml of OPTIMEM. After overnight incubation, cells were washed with PBS two times and growth medium (DMEM with 2% FBS as above) was added.

Transfection of these RNAs was associated with striking increases in SEAP secreted into the cell culture supernatant, as measured by assay of SEAP. SEAP was assayed using Tropix Phospha-Light Chemiluminescent Reporter Assay for secreted Alkine Phosphatase reagent (Tropix, Foster City, Calif.), according to the manufacturer's suggested protocol, but reduced ⅓ in scale. Luminescent signal detected by a TD-20/20 Luminometer (Turner Design).

The increase in SEAP occurred as a result of transfection with either MK0-Z or the replication deficient dS-MK0-Z RNA, indicating that the SEAP released in the initial weeks after transfection was expressed from the input RNA, not newly replicated RNA. High expression of SEAP was observed from 3′ETZ, reflecting greater transfection efficiency of this small RNA transcript. This experiment demonstrates the feasibility and utility of the SEAP cellular reporter system, and demonstrates the expression of Tat by the genetically modified HCV RNA.

Proof that infection had been accomplished by the transfection of MK0-Z RNA and that virus adaptation to replication in cultured cells had occurred under antibiotic selection pressure accumulated over the ensuring several months, as follows. FIG. 4 (left panel) shows the results of SEAP assays on media harvested from these cells during the first month after transfection with MK0-Z, and the pol(−) mutant dSMk0-Z. These cells were subsequently maintained in medium with a low concentration of fetal calf serum (2%) over the ensuing 3 months, during which the cells were split periodically and intermittently exposed to low concentrations of the antibiotic Zeocin as tolerated (about 10 to 25 μg/ml). There was no significant difference in cell survival in the presence of Zeo between cells transfected with MK0-Z, and those transfected with dSMK0-Z, but the former usually expressed somewhat higher levels of SEAP in the media (about 1.5 times to about 2 times higher than the control cells). At approximately 3 months, these cells (both MK0Z and ds-MKM0-Z transfected cells) underwent a spontaneous crisis with loss of viability. The supernatant fluids were collected and placed on replicate cultures of fresh Huh-SEAPo 10 cells in an attempt at blind passage of virus. Antibiotic selection was continued intermittently, with gradually intensifying Zeocin selection (intermittent exposure ultimately to 50 μg/ml). With the increase to 50 μg/ml Zeocin, sudden marked increases in SEAP expression were noted from replicate cultures of cells that had been inoculated with medium from the MK0-Z transfected cells, but not cells inoculated with the pol(−) mutant, dS-MK0-Z. This occurred about 7 months after the original transfection, and 4 months after the attempt at cell-free passage of virus. All cells were unable to survive the higher concentration of Zeo, however and the cultures were lost at this point. However, cells that had been previously frozen from the putative passage were recovered from the freezer, and subjected to intermittent concentrations of Zeocin ranging from 25-50 μg/ml. Results are shown in FIG. 5, and summarized in Table 2.

TABLE 2
Passage history of vMK0-Z-infected Huh-SEAP-o10 C-A
and C-B sublines.¹

| | Approximate | |
|---|---|---|
| | elapsed | |
| Passage | time (days) | Comments |
| P1 | 1 | Huh-SEAP-o10 cells transfected with |
| | | MK0-Z RNA, maintained in the absence of |
| | | antibiotic selection. |
| | 33 | Start intermittent Zeocin selection pressure, |
| | | 10-25 mg/ml. |
| | 75 | Cells entered crisis and were lost |
| P2 | 68 | Fresh Huh-SEAP-o10 cells infected with P1 day |
| | | 68 supernatant, and maintained in intermittent |
| | | Zeocin 25 mg/ml. |
| | 190 | Increase Zeocin to 25-50 mg/ml, with resulting |
| | | increase in SEAP expression. |
| | 197 | Cells frozen (continuously cultured cells lost |
| | | within about 1.5 months) |
| | 283 | Cells frozen on P2 day 197 were replated, |
| | | cultured in intermittent Zeocin 50-100 mg/ml, |
| | | with marked increase in SEAP expression. |
| | | P2 cells infected with P1 supernatant from |
| | | control dS-MK0-Z did not survive. |
| | 547 | Two cell lines (C-A and C-B), both established |
| | | on P2 day 283, maintained in intermittent |
| | | Zeocin 50-100 mg/ml with high SEAP. |
| P3 | 514 | Fresh Huh-SEAP-o10 cells infected with |
| | | 0.45 m-filtered supernatant media from P2 |
| | | C-A and C-B cell lines on day 544, |
| | | maintained in intermittent Zeocin 25 mg/ml. |

¹The term “vMK0-Z” is used to refer to the viral form of MK0-Z after passage.

As observed previously, striking increases occurred in the level of SEAP secreted from 12 of 12 replicate cultures of cells infected with medium from the MK0-Z-transfected cells, but not from any cultures of cells infected in parallel with medium from dS-MK0-Z transfected cells. Moreover, all of the control cell cultures were lost under exposure to 50 μg/ml Zeocin, while each of the cultures infected with MK0-Z material remained viable. Significantly, there was no increase in SEAP released into the medium from the dying cell lines (FIG. 5, dSma (C-A) and dSma (C-B)), consistent with the fact that all SEAP produced is actively secreted from the cells into the medium. This result confirms that cell death does not result in a false elevation of SEAP activity in culture supernatant fluids. The Zeocin resistance and SEAP expression displayed by these cells cannot be explained by fortuitous integration of DNA from the transfected material, since the cells shown in FIG. 5 were never transfected, only exposed to medium from transfected cells. Cell survival and SEAP expression also cannot be explained by cellular mutations in these experiments, as these events have occurred in multiple cultures exposed to the supernatant fluid of MK0-Z transfected cells, but not in related control cell cultures that were similarly exposed to media from dS-MK0-Z transfected cells.

Fluctuations in SEAP activity correlated in part with cell density, and cell viability. At times, these cultures demonstrated considerable cytopathology. However, it was demonstrated that there was minimal intracellular SEAP activity and that most SEAP is actively secreted from the cells. Thus, peaks of SEAP activity reflect peaks of SEAP synthesis, not release from dying cells.

The results shown in FIG. 5 indicate that these cells express two heterologous proteins encoded by MK0-Z, RNA. The Huh-SEAP-o 10 cells have acquired relative Zeocin resistance, indicating the expression of the Zeocin resistance protein, and they secrete 5- to 10-fold greater quantities of SEAP than control cells, indicating the expression of Tat. Moreover, RT-PCR has been used to successfully detect the presence of HCV RNA in samples of the supernatant fluids collected from these cells, using a primer set derived from the viral 5′NTR (see Example 5). Detection of the signal was dependent on Southern blotting of first round RT-PCR products, and amplification was dependent upon the inclusion of reverse transcriptase in the reaction. The results suggest that only small quantities of RNA are present, but confirm that the RT-PCR products are amplified from RNA and not contaminating DNA. The sequence of the amplified product was identical to the H77C strain 5′NTR, the virus from which the MK0-Z clone was derived. These results thus represent the first successful attempt at recovery of HCV from cells transfected with synthetic RNA.

One of the more important features of the experiment depicted in FIG. 5 is the significant change in the behavior of these HCV infected cells over the months of observation, both in terms of their increasing Zeocin resistance and increasing SEAP secretion. This is consistent with adaptation of the viral RNA to more efficient replication within these cells, as would be expected for a positive-strand RNA virus. Furthermore, since at this point all of the cells exposed to medium from cells transfected with the pol(−) mutant dS-MK0-7 have failed to survive Zeocin selection, it can now be assumed that all of the surviving cells harbor viral RNA. Thus, any further increases in SEAP expression must be indicative of greater abundance of the RNA and enhanced replication of the virus.

In summary, these two cell lines continue to demonstrate substantial Zeocin resistance and high level SEAP activity, two independent measures of protein expression from the second open reading frame of the modified vMK0-Z genome, more than 12 months after their infection with supernatant fluids taken from RNA-transfected cells. This is strong evidence of continued replication of the viral RNA in these cells.

### Example 5

### Passage of vMK0-Z to Fresh Huh-SEAP-o10 Cells

A third passage of vMK0-Z was carried out using supernatant media collected from the C-A and C-B cell lines on P2 day 540 (see Table 2). These media samples were passed through a 0.45μ filter and then used to feed fresh Huh-SEAP-o10 cells. Control cell cultures (n=6) were mock infected with normal media. One hundred and twenty hours after inoculation, these cells were exposed to intermittent Zeocin selection pressure (25 μg/ml). When treated with high concentrations of drug, or when maintained in continuous drug condition, these cells tend to die. Accordingly, drug exposure was intermittent, and not at high concentrations. The mock-infected cells were lost due to Zeocin toxicity by about day 546 (relative SEAP activity of infected to control cells at this point was 42658 and 31510, respectively, and is not shown in FIG. 6).

The results shown in FIG. 6 demonstrate the passage of SEAP expression activity and Zeocin resistance to fresh Huh-SEAP-o10 cells following inoculation of these cells with supernatant medium collected from vMK0-Z-infected cells.

### Example 6

### Detection of Viral RNA in Huh-SEAP-o10 Cell Lines

Despite the results described above, and the demonstration of viral antigen in MK0-Z infected cells (see Example 7), it has proven difficult to consistently demonstrate viral RNA in these cells. This Example describes methods for detecting the presence of viral RNA in Huh-SEAP-o10 cell lines.

Two different quantitative RT-PCR assays (LightCycler and TaqMan) have been used in recent efforts to detect viral RNA in lysates of the cells or in supernatant media. Greatest consistency of success has been in detection of viral RNA in supernatant media following PEG precipitation. This technique works very well, allowing concentration of 130 genome copies equivalent from 1 milliliter (ml) supernatant with 80% recovery. Viral RNA has been reproducibly but intermittently detected in the supernatant fluids; however, reliable detection of viral RNA in cell lysates has not been possible.

The primers and probes that have been used for these assays were as follows:

LightCycler RT-PCR

This method used the Lightcycler thermal cycler manufactured by Roche.

| | | |
|---|---|---|
| Primers: | | |
| Forward | 5′-GACACTCCACCATGAATCACT, nt 21 to 41, | (SEQ ID NO:10) |
| Reverse | 5′-GTTCCGCAGACCACTATGG, nt156 to 139, | (SEQ ID NO:11) |

Probes for Fluorescence Resonance Energy Transfer (FRET)

| | |
|---|---|
| 5′-AGAAAGCGTCTAGCCATGGCGTTAG(Fluor) | (SEQ ID NO:12) |
| 5′(LC640)ATGAGTGTCGTGCAGCCTCCAG(phosphate) | (SEQ ID NO:13) |

Briefly, the HCV virus was precipitated with PEG (Sigma, St. Louis, Mo.) prior to extraction with QIAamp serum kit Qiagen, Valencia, Calif.). Supernatant (1.3 ml) was mixed with 0.3 ml of 40% PEG and was placed in an ice bath for 4 hours. The mixture was then centrifuged at 10000×g for 30 minutes at 4° C. The supernatant was removed from the white pellet and 140 μl of TE was added to it. The RNA was then extracted from the viral pellet by following the manufacturers instructions. The eluate was treated with Dnase I as was instructed by the T7 mega transcription kit (Ambion), precipitated with 60 μg glycogen in 130 μl IPA, and stored at −80° C. The positive serum control was a volume of serum containing 5000 genome equivalents, added to media (1.3 ml TE) before precipitation with 0.3 ml PEG and extraction as discussed above. The HCV genome equivalents were determined by National Genetics Institute (Los Angeles, Calif.). The negative serum control was 1 μl of serum from an uninfected volunteer. The serum was treated in the same way as the positive control serum.

The single-tube RT-PCR reactions were carried out in capillary tubes in a reaction volume of 20 μl using the core reagents of RNA Amplification Kit Hybridization Probes (Roche). A 20 μl RT-PCR mixture contained 0.05 μM forward primer, 0.9 μM of reverse primer, RNA sample and 5 ul tube wash of purified sample RNA. The precipitated RNA was first reconstituted with RT-PCR master mix then was loaded into a glass capillary tube, after adding the 5 μl wash the tube was snap sealed with a plastic cap. The RT-PCR conditions were 55° C. for 15 minutes, 95° C. for 30 seconds, and 40 cycles of 94° C. for 0 seconds, 60° C. annealing for 15 seconds, and 72° C. extension for 15 seconds. The signal acquisition was at the end of the annealing step for 100 ms. After amplification was complete, a melting curve was performed by cooling to 55°, holding at 55° C. for 30 seconds, and then heating slowly at 0.2 C./seconds until 90° C. Signal was collected continuously during this melting to monitor the dissociation of the 5′-LC640-labeled probe. The melting curves were then displayed as—F/d T vs T plots by LightCyler software version 3.

Results obtained in the LightCycler assay with PEG-precipitated supernatant media collected from the C-A and C-B cell sublines are shown in FIG. 7, which shows the melting curve detected by the FRET method. The melting curve indicates the specificity of product. Both C-A and C-B's curve matches that of positive control. The height of the curve correlates with the amount of the product produced. The negative media control was cell culture media maintained in the isolation room in which the C-A and C-B cell sublines are maintained. The negative serum control was contributed by a volunteer.

TaqMan RT-PCR

Primers (see Takeuchi et al., Gastroenterol., 116, 636-642 (1999)):

| | | |
|---|---|---|
| Forward | 5′-CGGGAGAGCCATAGTGG | (SEQ ID NO:14) |
| Reverse | 5′-AGTACCACAAGGCCTTTCG | (SEQ ID NO:15) |

| | |
|---|---|
| TaqMan probe: | |
| 5′-(FAM)-CTGCGGAACCGGTGAGTACAC(TAMRA)-3′ | (SEQ ID |
| | NO:16) |

RNA was obtained from cells as described above for PCR with the Lightcycler thermal cycler. This experiment was set up according to the protocol provided in TaqMan EZ RT-PCR Core Reagents Protocol (product number 402877, Applied Biosystems, Foster City, Calif.). Briefly, All single-tube EZ RT-PCR reactions were carried out in optical MicroAmp reaction tubes with optical lids and in 50 μl volume in a 96-well format. The RNA amplification contained 1×amplification buffer, 3 mM manganese, 0.5 Units (U) AmpErase uracil-N-glycosylate, 7.5 U rTth DNA polymerase, RNA, 200 nM forward and reverse primers, 200 μM each dNTP, 500 μM of d UTP. ABI7700 Sequence Detector version 1.6.3 software was used for sample analysis. Thermocycling conditions were one cycle at 50° C. for 2 minutes, one cycle at 60° C. for 30 minutes, one cycle at 95° C. for 5 minutes, 40 cycles at 95° C. for 20 seconds and 60° C. for 1 minutes.

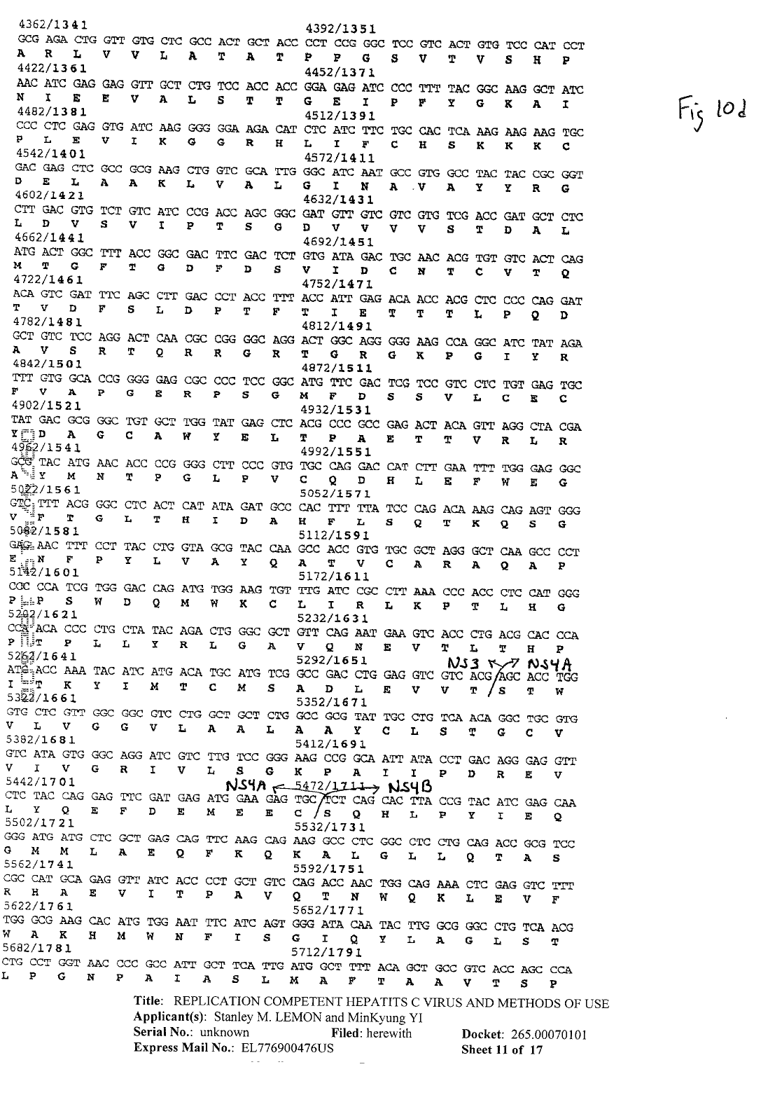

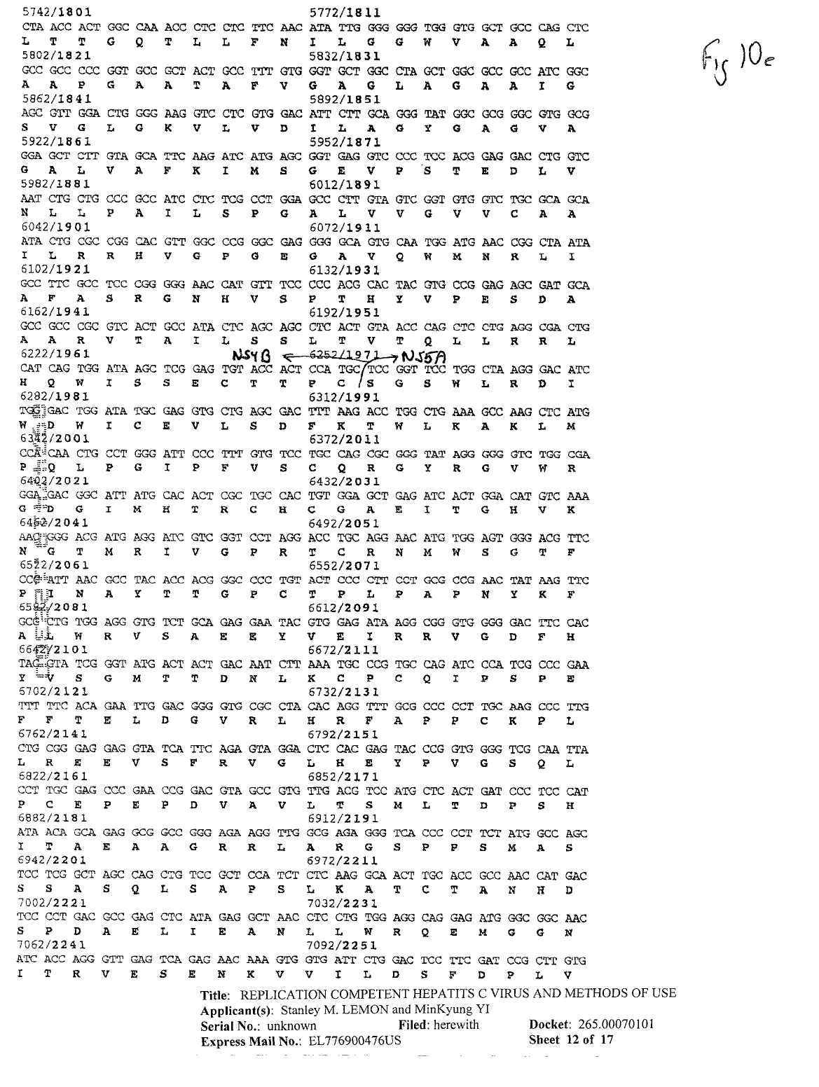

FIG. 8 shows results of TaqMan RT-PCR The C-A and C-B product as detected according to program is aligned along with a known concentration of positive control HCV. The approximate number of HCV protracted from this graph is shown in Table 3.

TABLE 3
TaqMan quantitation of HCV RNA in supernatant media.

| Supernatant from: | Number of genome equivalents |
|---|---|
| Positive serum control (5000 ge¹) | 4188 |
| C-B | 109 |
| C-A | 136 |
| C-B (unhealthy culture)² | 3 |
| C-A (unhealthy culture)² | 7 |
| Negative control media | 24³ |
| Medium | 0 |
| Negative control | 0 |

¹ge, genome equivalents.
²Cultures were losing viability.
³This is believed to be the result of contamination.

There was good correlation between the TaqMan and LightCycler results on these specimens.

### Example 7

### Demonstration of Viral Antigens in vMK0-Z-infected Huh-SEAP-o10 Cell Lines

Viral antigens expressed from both coding regions (i.e., the coding region encoding the viral polypeptides and the coding region inserted in the 3′ NTR) in the modified HCV genome have been demonstrated in vMK0-Z infected Huh-SEAP-o10 cells by indirect immunofluorescence. Negative controls for these experiments were uninfected Huh-SEAP-o10 cells. Cells were grown in tissue culture chamber slides and fixed in acetone-methanol at room temperature prior to staining. Cells were fixed in 50% methanol/50% Acetone for 10 minutes. Blocking agent was 3% BSA in PBS. The primary antibodies used were a mouse monoclonal antibody against HCV core protein, (anti-core antibody, provided by Johnson Lau, Schering-Plough Research Institute, Kennilworth, N.J.) used at a dilution of 1:100, a rabbit polyclonal antibody raised against Sh Ble protein (anti-Zeo antibody, CAYLA, France) used at a dilution of 1:250. The secondary antibodies were fluorescene conjugated anti-mouse or anti-rabbit. Antibodies were incubated with cells for 1 hour each. Between each incubation, the cells were washed three times for 5 minutes each with PBS. Nuclear counterstain was done using DAPI. Dapi staining to detect nucleus was done in 1:10,000 dilution in PBS. It was incubated for 5 minutes, followed by three washes for 5 minutes each in PBS. Photographic exposure times and contrast enhancements were identical for the infected cells and control cell images.

Exposure of cells to an anti-core antibody demonstrated the presence of HCV core protein in vMK0-Z infected cells. Exposure of cell to an anti-zeocin resistance protein demonstrated the presence of the Zeocin resistance protein in vMK0-Z infected cells.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A replication competent hepatitis C virus comprising a hepatitis C virus genome and a heterologous polynucleotide, wherein the hepatitis C virus genome comprises a 3′ non-translated RNA and the heterologous polynucleotide is present in the 3′ non-translated RNA.

2. The replication competent hepatitis C virus of claim 1 wherein the heterologous polynucleotide is present in the variable region of the 3′ non-translated RNA.

3. The replication competent hepatitis C virus of claim 1 wherein the heterologous polynucleotide comprises a coding sequence.

4. The replication competent hepatitis C virus of claim 4 wherein the coding sequence encodes a selectable marker or a detectable marker.

5. The replication competent hepatitis C virus of claim 4 wherein the selectable marker encodes resistance to an antibiotic selected from the group consisting of neomycin and phleomycin D1.

6. The replication competent hepatitis C virus of claim 3 wherein the coding sequence encodes an immunogenic polypeptide.

7. The replication competent hepatitis C virus of claim 3 wherein the coding sequence encodes a fusion polypeptide.

8. The replication competent hepatitis C virus of claim 3 further comprising a regulatory region operably linked to the coding sequence.

9. The replication competent hepatitis C virus of claim 8 wherein the regulatory region comprises an internal ribosome entry site.

10. The replication competent hepatitis C virus of claim 3 wherein the coding sequence encodes a transactivator.

11. The replication competent hepatitis C virus of claim 10 wherein the transactivator comprises a tat polypeptide.

12. The replication competent hepatitis C virus of claim 11 wherein the tat polypeptide comprises SEQ ID NO:19.

13. The replication competent hepatitis C virus of claim 1 wherein the heterologous polypeptide comprises a first coding sequence encoding a transactivator, a second coding sequence encoding a selectable marker, and a third coding sequence encoding a cis-active proteinase, the third coding sequence located between the first and second coding sequences, and the first, second, and third coding sequences together encoding a fusion polypeptide.

14. The replication competent hepatitis C virus of claim 13 wherein the cis-active proteinase comprises amino acids SEQ ID NO:30.

15. The replication competent hepatitis C virus of claim 1 wherein the virus is isolated.

16. The replication competent hepatitis C virus of claim 1 wherein the virus replicates in vitro in a human cell.

17. The replication competent hepatitis C virus of claim 16 wherein the cell is a cultured cell.

18. The method of claim 17 wherein the cultured cell is a hepatocyte.

19. The method of claim 18 wherein the hepatocyte is a human hepatocyte.

20. The replication competent hepatitis C virus of claim 1 wherein the virus replicates in vivo.

21. The replication competent hepatitis C virus of claim 1 wherein the virus has a genotype selected from the group consisting of 1a, 1b, 1c, 2a, 2b, 2c, 3a, 3b, 4, 5a, and 6a.

22. The replication competent hepatitis C virus of claim 1 wherein the virus is an RNA polynucleotide.

23. The replication competent hepatitis C virus of claim 22 wherein the RNA polynucleotide is present in a viral particle.

24. The replication competent hepatitis C virus of claim 1 wherein the virus is a DNA polynucleotide.

25. The replication competent hepatitis C virus of claim 24 wherein the DNA polynucleotide is present in a vector.

26. A cell comprising the replication competent hepatitis C virus of claim 1.

27. The cell of claim 26, wherein the cell is a cultured cell.

28. A method for modifying a hepatitis C virus comprising a 3′ non-translated RNA, the method comprising inserting a heterologous polynucleotide into the 3′ non-translated RNA.

29. The method of claim 28 wherein the heterologous polynucleotide is present in the variable region of the 3′ non-translated RNA.

30. The method of claim 28 wherein the heterologous polynucleotide comprises a coding sequence.

31. The method of claim 30 wherein the coding sequence encodes a selectable marker or a detectable marker.

32. The method of claim 31 wherein the selectable marker encodes resistance to an antibiotic selected from the group consisting of neomycin and phleomycin D1.

33. The method of claim 30 wherein the coding sequence encodes an immunogenic polypeptide.

34. The method of claim 30 wherein the coding sequence encodes a fusion polypeptide.

35. The method of claim 30 further comprising a regulatory region operably linked to the coding sequence.

36. The method of claim 35 wherein the regulatory region comprises an internal ribosome entry site.

37. The method of claim 30 wherein the coding sequence encodes a transactivator.

38. The method of claim 37 wherein the transactivator comprises a tat polypeptide.

39. The method of claim 38 wherein the tat polypeptide comprises SEQ ID NO:19.

40. A method for selecting a replication competent hepatitis C virus, the method comprising:
incubating a cell in the presence of a selecting agent, wherein:
the cell comprises a hepatitis C virus comprising a heterologous polynucleotide;
the heterologous polynucleotide comprises a coding sequence encoding a selectable marker that confers resistance to the selecting agent; and
the selecting agent inhibits replication of a cell that does not express the selectable marker; and
detecting the presence or absence of a cell that replicates in the presence of the selecting agent, wherein the presence of such a cell indicates the hepatitis C virus is replication competent.

41. The method of claim 40 wherein the cell is a first cell, the method further comprising:
isolating a virus particle produced by the first cell;
exposing a second cell to the isolated virus particle and incubating the second cell in the presence of the selecting agent; and
detecting the presence or absence of a second cell that replicates in the presence of the selecting agent, wherein the presence of such a cell indicates the hepatitis C virus present in the first cell produces an infectious virus particle.

42. An virus particle isolated according to the method of claim 41.

43. The method of claim 41 wherein isolating the virus particle comprises removing cells from media in which the first cell is incubated.

44. The method of claim 40 wherein the heterologous polynucleotide is present in the variable region of the 3′ non-translated RNA.

45. The method of claim 40 wherein the selecting agent is an antibiotic.

46. The method of claim 45 wherein the selectable marker encodes resistance to an antibiotic selected from the group consisting of neomycin and phleomycin D1.

47. The method of claim 40 wherein the heterologous polynucleotide further comprises a regulatory region operably linked to the coding sequence.

48. The method of claim 47 wherein the regulatory region comprises an internal ribosome entry site.

49. The method of claim 40 wherein the cell is a cultured cell.

50. The method of claim 49 wherein the cultured cell is a hepatocyte.

51. The method of claim 50 wherein the hepatocyte is a human hepatocyte.

52. The method of claim 40 wherein the hepatitis C virus has a genotype selected from the group consisting of 1a, 1b, 1c, 2a, 2b, 2c, 3a, 3b, 4, 5a, and 6a.

53. A method for detecting a replication competent hepatitis C virus, the method comprising:
incubating a cell comprising a hepatitis C virus, wherein:
the hepatitis C virus comprises a heterologous polynucleotide comprising a coding sequence encoding a transactivator;
the cell comprises a transactivated coding region and an operator sequence operably linked to the transactivated coding region; and
the transactivated coding region encodes a detectable marker,
wherein the transactivator binds to the operator sequence; and
detecting the presence or absence of the detectable marker, wherein the presence of the detectable marker indicates the cell comprises a replication competent hepatitis C virus.

54. The method of claim 53 wherein the heterologous polynucleotide is present in the variable region of the 3′ non-translated RNA.

55. The method of claim 53 wherein the heterologous polynucleotide further comprises a coding sequence encoding a selectable marker, the coding sequence encoding the transactivator and the coding sequence encoding the selectable marker together encoding a fusion polypeptide.

56. The method of claim 55 wherein the heterologous polynucleotide further comprises a coding sequence encoding a cis-active proteinase present between the coding sequence encoding the selectable marker and the coding sequence encoding the transactivator.

57. The method of claim 55 further comprising a regulatory region operably linked to the coding sequences encoding the fusion polypeptide.

58. The method of claim 57 wherein the regulatory region comprises an internal ribosome entry site.

59. The method of claim 53 wherein the transactivator comprises a tat polypeptide.

60. The method of claim 59 wherein the tat polypeptide comprises SEQ ID NO:19.

61. The method of claim 55 wherein the fusion polypeptide further comprises a cis-active proteinase.

62. The method of claim 55 wherein the hepatitis C virus has a genotype selected from the group consisting of 1a, 1b, 1c, 2a, 2b, 2c, 3a, 3b, 4, 5a, and 6a.

63. The method of claim 55 wherein the cell is a cultured cell.

64. The method of claim 63 wherein the cultured cell is a hepatocyte.

65. The method of claim 64 wherein the hepatocyte is a human hepatocyte.

66. The method of claim 55 further comprising isolating the replication competent hepatitis C virus from the cell.

67. The method of claim 55 wherein the detectable marker is secretory alkaline phosphatase.

68. A method for identifying a compound that inhibits replication of a hepatitis C virus, the method comprising:
contacting a cell comprising a replication competent hepatitis C virus with a compound, the replication competent hepatitis C virus comprising a heterologous polynucleotide;
incubating the cell under conditions allowing replication of the replication competent hepatitis C virus in the absence of the compound; and
identifying the presence or absence of the replication competent hepatitis C virus, wherein a decrease in the presence of replication competent hepatitis C virus in the cell contacted with the compound relative to the presence of replication competent hepatitis C virus in a cell not contacted by the compound indicates the compound inhibits replication of the replication competent hepatitis C virus.

69. The method of claim 68 wherein identifying comprises selecting the presence or absence of the replication competent hepatitis C virus.

70. The method of claim 68 wherein identifying comprises detecting the presence or absence of the replication competent hepatitis C virus.

71. The method of claim 70 wherein the heterologous polynucleotide comprises a first coding sequence encoding a transactivator, and the cell comprising a polynucleotide comprising a transactivated coding sequence encoding a detectable marker and an operator sequence operably linked to the transactivated coding sequence, wherein the transactivator binds to the operator sequence and alters expression of the transactivated coding sequence, and wherein identifying the presence of replication competent hepatitis C virus in the cell comprises detecting the detectable marker encoded by the transactivated coding sequence.

72. The method of claim 71 further comprising introducing to the cell the replication competent hepatitis C virus before contacting the cell with the compound, wherein the cell is contacted with the compound before, at the same time, or after introducing the replication competent hepatitis C virus to the cell.

73. A vector comprising a cDNA of a replication competent hepatitis C virus comprising a hepatitis C genome and a heterologous polynucleotide, the hepatitis C virus genome comprising a 3′ non-translated RNA, the heterologous polynucleotide present in the 3′ non-translated RNA.

74. The vector of claim 73 wherein the heterologous polypeptide comprises a coding sequence.

75. The vector of claim 73 wherein the heterologous polypeptide further comprises a regulatory region operably linked to the coding sequence.

76. The vector of claim 75 wherein the regulatory region comprises an internal ribosome entry site.

77. The vector of claim 73, the vector further comprising a promoter operably linked to the replication competent hepatitis C virus.

78. An RNA molecule produced by the vector of claim 77, the RNA molecule comprising the replication competent hepatitis C virus.

79. A cell comprising the vector of claim 73.
